(19) United States
(12) Patent Application Publication
(10) Pub. No.: US 2007/0254049 A1
(43) Pub. Date: Nov. 1, 2007

(54) Method and feed for increasing the efficiency of ruminary production

(76) Inventors: James Frederick Beck, Brooklyn Park, MN (US); Michael S. Allen, Okemos, MI (US); Rick Grant, Lincoln, NE (US); Charles Todd Milton, Dannebrog, NE (US); William P. Weiss, Shreve, OH (US)

Correspondence Address: GLEN E. SCHUMANN;C/O MOSS & BARNETT, 90 SOUTH SEVENTH STREET, 4800 WELLS FARGO CENTER, MINNEAPOLIS, MN, 55402-4129, US

(21) Appl. No.: 11/414,636

(22) Filed: Oct. 5, 2006

Related U.S. Application Data
(63) Continuation of application No. 10/477,863, filed on Nov. 13, 2003, now abandoned.
(60) Provisional application No. 60/294,926, filed on May 31, 2001.
(60) Provisional application No. 60/348,150, filed on Jan. 14, 2002.

(51) Int. Cl. A61K 36/18

(52) U.S. Cl. 424/725

(57) ABSTRACT

The present invention relates to a method and for enhancing productivity of ruminant animals. Particularly, a method comprising determining particular corn hybrids of specific endosperm type and NDF content for use as silage and/or a grain supplement and combining other diet components to form a feed ration that optimizes the site of starch digestion, and a feed made using the method of the invention.

The present invention relates to a method and feed for enhancing productivity of ruminant animals. Particularly, the present invention is a method comprising selecting corn hybrids of specific endosperm type and NDF content for use as a silage and/or a grain supplement and combining other diet components to form a feed ration that optimizes the site of starch digestion, and a feed ration developed using the method of the invention.

Corn plants are harvested as whole plants and preserved by ensiling (corn silage) or are harvested for grain only. Starch is located in the endosperm fraction of grain and its digestibility is variable and affected by type of endosperm. There are two types of endosperm: floury and vitreous. Floury endosperm is more rapidly and completed digested than vitreous endosperm, which must be adjusted for in ration formulation for optimum performance. For ruminant animals, starch is initially fermented in the rumen by microbes to short-chain fatty acids. The fraction that passes from the rumen escaping fermentation can be digested in the abomasums (gastric stomach) and absorbed as glucose in the small intestine. That which passes from the small intestine, can be fermented by microbes in the large intestine. The fraction of starch fermented in the rumen has been shown to have large effects on feed intake and animal production such as milk yield. For example, starch comprises 20 to 35% of dairy cow diets and ruminal degradation of starch ranges from less than 40% to more than 90% of total starch. Starch degraded by microbes in the rumen supplies energy to the cow as short-chain fatty acids such as acetic, propionic, and butyric acids, and energy to microbes to grow and produce microbial protein. Microbial protein produced in the rumen is the major source of protein for the cow and is of high biological value with an amino acid profile most similar to milk. Inadequate ruminally degraded starch will result in less energy to the cow in the form of short-chain fatty acids and less microbial protein available to the cow. Conversely, excess ruminally degraded starch will result in ruminal acidosis because acid production by microbes in the rumen exceeds the ability of the cow to absorb and utilize the acids. Ruminal acidosis is a common problem for ruminants and results in decreased feed intake, lower fiber digestibility, low efficiency of microbial protein production, as well as ruminal ulcers, liver abscesses, laminitis (sore feet), and displaced abomasums (twisted stomach). In a recent review, Allen (2000) showed that approximately one-third of the studies reported in the literature in which ruminal degradability of starch source is increased, resulted in approximately a 20% decrease in feed intake. A recent study by Oba and Allen (2000) shows that starch source with higher ruminal degradability results in lower feed intake, milk yield, and microbial efficiency than a starch source with higher ruminal digestibility.

Forages such as alfalfa, grass, and corn silage are fed to ruminants to provide fiber necessary to provide an optimal ruminal environment for starch degradation and energy utilization. However, silages comprised of these forages are extremely variable in composition and fermentability. Silages are filling and have inadequate energy for optimal animal production such as high milk yield in dairy cows or weigh gain in cattle. Thus, forages such as corn silage are often supplemented with supplements including cereal grains for energy. Corn is the major cereal grain, typically in combination with a silage, fed to ruminants in much of the U.S. Ruminal digestibility of corn grain varies with conservation method, processing such as grinding or steam flaking, and endosperm type.

Corn is conserved (harvested and stored until feeding) as high moisture corn or as dry corn. High moisture corn is harvested as the corn dries to the desirable range of 24% to 30% moisture content although the range is much greater in practice. The harvested high-moisture corn is then stored in silos, pits, or plastic bags where it ferments. This process is called ensiling and the acides produced by fermentation preservies the corn (inhibits microbes such as yeasts and molds that cause spoilage). Alternatively, corn can be left in the field to dry further (<15% moisture) and stored dry. Dry corn is not prone to spoil because it is too dry for significant microbial activity. The two conservation methods vary by both moisture content of corn and by the ensiling process.

Corn silage is a major source of forage neutral detergent fiber (NDF) and NB_{L} in many diets. Increasing the concentration of NDF in corn silage would mean that less NDF would have to be grown or purchased by the dairy farmer. Thus, corn silage hybrids with higher than normal NDF concentrations could have economic value as a fiber source. However, that value would be reduced or eliminated if the higher NDF concentration resulted in lower digestibility and lower available energy concentrations. Thus, there is a need for methods and feed compositions comprised of optimal combinations of silages and cereal grains to maximize animal production and animal health.

Thus, the present invention is directed to corn hybrids of selected endosperm types for use as corn silage and/or supplemental grain in feed rations for the purpose of optimizing the ruminal environment for starch degradation and energy utilization.

The present invention is also directed to a method of selecting one or more corn hybrids of a specific endosperm type for use as silage and/or supplemental grain, and combining the selected hybrid with other components to obtain an optimal ruminal environment for starch degradation and energy utilization.

The present invention is further directed to a method of selecting one or more corn hybrids with high NDF concentration for use as silage, and combining the high NDF hybrid with a supplemental grain comprised of a floury endosperm hybrid to obtain an optimal ruminal environment for starch degradation and energy utilization.

The present invention includes corn hybrids selected for endosperm types for use as corn silage and/or supplemental grain in feed rations for the purpose of optimizing the ruminal environment for starch degradation and energy utilization. The present invention is also directed to a method of selecting one or more corn hybrids of a specific endosperm type for use as silage and/or supplemental grain, and combining the selected hybrid with other components to obtain an optimal ruminal environment for starch degradation and energy utilization. Supplemental grain may be prepared according to the invention by grind size, method of conservation, moisture content, or whether such grain is steam flaked. Also, the present invention is further directed to a method of selecting one or more corn hybrids with high NDF concentration for use as silage, and combining the high NDF hybrid with a supplemental grain comprised of a floury endosperm hybrid to obtain an optimal ruminal environment for starch degradation and energy utilization.

The present invention therefore provides:

A method for enhancing production comprising the steps of: a) selecting a corn hybrid for use as supplemental grain; b) preparing said supplemental grain comprising said corn hybrid; c) determining the endosperm type of said corn hybrid; d) selecting silage to combine with said supplemental grain to form a feed ration for a ruminant; and e) determining the amounts of said silage and said supplemental grain for said feed ration, wherein when said feed ration is consumed by the ruminants, said amounts optimize the ruminal environment for starch degradation and energy utilization. Preferably, the supplemental grain is prepared from hybrid corn having a high moisture content, or is prepared using corn hybrid having dry grain, or is prepared using hybrid corn having been steam flaked, or is prepared based on particle size. Preferably, the endosperm of the corn hybrid is floury or vitreous.

In another preferred embodiment, the ruminant is a dairy cow or a steer.

In yet another preferred embodiment, a corn hybrid is corn hybrid N48-V8. In yet another preferred embodiment supplemental grain is comprised of the seed of corn hybrid NX7219.

The present invention further provides:

A method for enhancing ruminant production comprising the steps of: a) selecting a first corn hybrid for floury endosperm type for use as supplemental corn grain; b) preparing a supplemental corn grain comprising said first corn hybrid; c) selecting a second corn hybrid with high NDF, low starch, content for use as corn silage; d) preparing corn silage comprising said second corn hybrid; and e) combining said supplemental grain, said corn silage, and other components to form a feed ration, wherein said supplemental grain and said corn silage are combined in amounts to optimize the ruminal environment for starch degradation and energy utilization. Preferably, a corn hybrid selected for use as corn silage has a low NDF, high starch, content. Preferably, a corn hybrid selected for use as corn silage has high NDF digestibility.

Corn silage and supplemental grain supply the major energy, starch, and fiber component of a feed ration for the ruminant animal. For example, grain is the major energy source in feedlot finishing diets, typically comprising 80% or more of the diet dry matter. Improvements in the feeding value of corn grain can have a significant impact on cattle performance.

Grain processing methods that increase the amount of starch digested in the rumen of feedlot cattle consistently result in improved feed efficiencies. However, processes like steam flaking cost between $5 and $15 per ton, and are very energy intensive. Additionally, for both feedlot cattle and dairy cows, over processing of corn can result in acidosis challenges throughout the feeding period resulting in reduced feed intake, daily gain, and feed efficiency. Thus, corn hybrids that are naturally more digestible in the rumen could increase the challenge of managing acidosis when fed as steam flaked or high-moisture corn. Thus, the invention is directed to developing feed rations having the optimum combination of floury or vitreous endosperm hybrids and supplemental components for optimizing the ruminal environment for starch digestion, energy utilization, and ruminant performance. High NDF content hybrids are also used to optimize the ruminal environment for starch digestion, energy utilization, and ruminant performance.

The invention will be further understood with reference to the following illustrative embodiments, which are purely exemplary and should not be taken as limiting the true scope of the present invention as described in the claims.

### EXAMPLE I

### Effects of Starch Endosperm Type and Corn Processing Method on Feedlot Performance, Dietary Net Energy Values, Ruminal Fermentation, and Feed Intake Behavior in Finishing Cattle Fed High-Grain Diets

Corn Grain Production, Hybrids, Harvest, and Processing: Two hybrids are planted and grown under irrigation in a similar field to represent differences in endosperm type. One hybrid had primarily flinty endosperm, Cargill 6409 (C6409) and the other floury, Novartis 7219 (N7219). Grain is harvested as high-moisture corn or dry corn. High-moisture harvest is conducted when the corn grain reached 28-30% moisture. The high-moisture corn is coarsely rolled and stored in silo bags until feeding. Dry grain is harvested at approximately 18% moisture and dried to 15% moisture for storage.

Finishing Performance Trial: One hundred sixty steer calves (average initial weight 642 lb) are randomly allotted to one of 16 pens (10 head/pen; four replications/treatment) so that the average initial weight is similar among pens. Treatments are arranged as a 2×2 factorial design: N7219 fed as either fed as either high-moisture or dry-rolled corn and C6409 fed as either high-moisture or dry-rolled corn. The finishing diets (Table 1) are formulated to contain equal amounts of forage, crude protein, vitamins, minerals and feed additives (Rumensin @ 30 grams/ton). Because N7219 contained less crude protein than C6409 (8.74 versus 10.1%, respectively), corn gluten meal is supplemented to N7219 diets so that all diets contained similar amounts of protein from corn grain. Steers are implanted with Synovex C on day 1 and reimplanted on day 72 with Revalor-S. Steers are fed 191 days, and stepped up to their final finishing diets in 28 days using a series of transition diets containing 45, 35, 25 and 15% alfalfa hay (dry-matter basis) fed for 7 days each. Treatment grain sources supplied all of the corn grain during the ration step-up period.

Initial weights are the average of two consecutive early morning weights taken prior to feeding. Final weights are calculated by dividing hot carcass weight by a 63% dressing percentage. At slaughter, hot carcass weight, carcass fat thickness, marbling score, USDA Quality Grade and Yield grade are measured. Feedlot performance and carcass data are analyzed as a completely randomized design using the GLM procedure of SAS. Pen is the experimental unit. Orthogonal contrasts are used to test the main effects of corn hybrid and grain processing and the interaction of corn hybrid by grain processing.

Ruminal Metabolism Trial: A 4×4 Latin square is conducted using ruminally fistulated steers to measure the effects of endosperm type fed as either dry-rolled or high-moisture corn on nutrient digestibility and ruminal fermentation characteristics. The metabolism trial is conducted simultaneously with the feedlot performance trial, and uses the same diets. Each of the four experimental periods consisted of 24 days: 14-day diet adaptation and 10-day continuous ruminal pH measurements. Feed intake is measured continuously throughout each period. Ruminal fluid samples are collected on days 23 and 24 of each period at 0, 3, 6, 9, 12, 18, and 24 hours post feeding, and analyzed for volatile fatty acid and ruminal ammonia concentrations. Fecal grad samples are taken on days 20 through 24 four times daily at 6-hour intervals, with collection time advanced 1.5 hour each day, such that samples are obtained over each 1.5-hour interval of a 24-hour cycle. Feed ingredient and fecal samples are dried in a 50 degree C. oven and analyzed for dry matter, organic matter and starch to calculate total tract digestibility. Chromic oxide is used as an indigestible maker for estimating fecal output.

Average ruminal pH, ruminal pH change and variance, and area of ruminal pH below 5.6 are calculated for each day of continual data acquisition. The 10 individual days are averaged for each animal within each period, and this data is used for analysis.

Digestibility and ruminal pH data are analyzed using the GLM procedure of SAS. The model included treatment, animal and period. Orthogonal contrasts are used to test the effects of grain processing, grain hybrid and the interaction between grain processing method and hybrid. Ruminal volatile fatty acids (VFA) and ammonia data are analyzed using the MIXED procedure of SAS. The model included treatment, time, animal and period. Orthogonal contrasts are used to test the effects of grain processing, grain hybrid and the interaction between grain processing method and hybrid.

Results of the Finishing Performance Trial: Dry matter intake is similar between hybrids and grain processing treatments (Table 2). Interactions are observed between hybrid and grain processing method for daily gain (P=0.08), feed efficiency (P=0.01), hot carcass weight (P=0.09) and fat thickness (P=0.07). Daily gain is similar between hybrids when fed as high-moisture corn and hybrid N7219 fed as dry-rolled corn. Feeding C6409 as dry-rolled corn resulted in a 8.1% reduction in daily gain compared with the other treatments. When fed as dry-rolled corn, hybrid 7219 resulted in a 0.27 lb/d increase in a daily gain or a total of 51 lb of gain for the feeding period.

The interaction observed between hybrid and processing method for feed efficiency is a result of magnitude of change between feeding each hybrid as high-moisture of dry-rolled corn. Feed efficiencies are similar between hybrids when fed as high-moisture corn (Table 2). The amount of feed required to support each pound of gain increased 3.4% (P<0.10) when hybrid N7219 is fed as dry-rolled compared with high-moisture corn, whereas, the amount of feed required per pound of gain increased 8.7% (P<0.10) when hybrid C6409 is fed as dry-rolled versus high-moisture corn. The response to more intensive processing is much greater for hybrid C6409 compared with hybrid N7219. The response to processing method is 2.6 times greater with hybrid C6409 compared with N7219. Because the interaction for feed efficiency is a magnitude of difference between hybrids, the main effects of hybrid and processing method is discussed. Averaged across grain hybrids, feeding high-moisture corn improved feed efficiency 6.1% (P<0.01) compared with dry-rolled corn. Averaged across grain processing method, feeding N7219 improved feed efficiency 3.1% (P<0.01) compared with C6309.

Differences among treatments in hot carcass weight are a reflection in daily gain (Table 2). Fat thickness is similar between hybrids when fed as high-moisture corn and hybrid N7219 fed as dry-rolled corn. Fat thickness is lower in steers fed dry-rolled hybrid C6409 (P<0.10) compared with other treatments. Marbling scores are higher (P=0.03) for steers fed high-moisture compared with dry-rolled corn. Other carcass characteristics are similar among treatments. Differences in carcass characteristics reflect differences in energy values associated with hybrid and grain processing methods.

Results from the Metabolism Trial: Results from the metabolism trial are presented in Tables 3, 4 and 5. Dry matter intake is similar between hybrid and grain processing method (Table 3). Total tract dry matter digestibility tended (P=0.12) to be higher for steers fed hybrid N7219 compared with C6409. Organic matter (P=0.10) and starch (P=0.09) are higher for steers fed hybrid N7219 compared with C6409. This is a result of the lower protein and higher starch content of N7219 compared with C6409. Total tract organic matter (trend; P=0.15) and starch digestibility (P=0.05) are higher for hybrid N7219 compared with C6409. The differences observed with total tract nutrient digestibility support the improved animal performance observed with hybrid N7219 versus C6409.

Ruminal pH measurements are not influenced by grain hybrid (Table 4). Feeding high-moisture corn resulted in larger (P=0.07) changes (maximum-minimum) and greater (P=0.08) variance in daily ruminal pH. This should be expected since the rate and extent of ruminal starch digestion should have been greater for high-moisture compared with dry-rolled corn. Total VFA and acetate concentrations are similar between treatments (Table 5). Molar proportions of propionate are higher (P=0.01) in steers fed high-moisture versus dry-rolled corn, and higher in steers fed hybrid N7219 compared with C6409. Other ruminal VFA concentrations are similar among treatments. Larger proportions of the total VFA concentration as propionate for high-moisture corn and hybrid N7219 support the improvements in feed efficiency observed in the animal performance experiment.

Ruminal ammonia concentration is lower (P=0.07) for steers fed high-moisture corn compared with those fed dry-rolled corn (Table 5). Lower ruminal ammonia concentrations suggest that more starch is being ruminally digested with high-moisture corn compared with to dry-rolled corn. Ruminal ammonia concentrations are similar between grain hybrids.

TABLE 1
Experimental Diet Composition (100% Dry Matter Basis).

| | Dietary Ingredients | | | | |
|---|---|---|---|---|---|
| | Dry- | High- | | | |
| | Rolled | Moisture | Alfalfa | Cane | Milled |
| Diet | Corn | Corn | Hay | Molasses | Supplement |
| N7219 | — | 81.0 | 8.0 | 3.0 | 8.0 |
| N7219 | 81.0 | — | 8.0 | 3.0 | 8.0 |
| C6409 | — | 81.0 | 8.0 | 3.0 | 8.0 |
| C6409 | — | 81.0 | 8.0 | 3.0 | 8.0 |

TABLE 2
Effect of corn hybrid and processing method on performance and carcass
characteristics of finishing steers.

| | 7219 | | 6409 | | | Contrasts^{a} | | |
|---|---|---|---|---|---|---|---|---|
| Item | HMC | DRC | HMC | DRC | SEM | Process | Hybrid | Inter |
| Initial wt., lb | 641 | 642 | 642 | 643 | 1.3 | — | — | — |
| Final wt., lb | 1337^{c} | 1333^{c} | 1332^{c} | 1281^{d} | 12.6 | .05 | .04 | .09 |
| Days on feed, n | 191 | 191 | 191 | 191 | — | — | — | — |
| Dry matter | 19.54 | 20.05 | 19.38 | 19.59 | .25 | .16 | .23 | .55 |
| intake, lb/d | | | | | | | | |
| Daily gain, lb | 3.65^{c} | 3.61^{c} | 3.61^{c} | 3.34^{d} | .06 | .03 | .03 | .08 |
| Feed efficiency | 5.36^{c} | 5.55^{d} | 5.37^{e} | 5.88^{f} | .05 | <.01 | <.01 | .01 |
| Hot carcass wt., | 843^{c} | 840^{c} | 839^{c} | 807^{d} | 7.9 | .05 | .04 | .09 |
| lb | | | | | | | | |
| Fat thickness, in. | .43^{c} | .41^{c} | .45^{c} | .33^{d} | .02 | .01 | .22 | .07 |
| Yield grade | 2.31 | 2.25 | 2.23 | 1.90 | .13 | .17 | .13 | .35 |
| Marbling score^{b} | 4.47 | 4.39 | 4.59 | 4.19 | .09 | .03 | .69 | .11 |
| Percent Choice | 20.5 | 17.5 | 22.5 | 18.0 | 3.7 | .33 | .74 | .85 |
| Percent Select | 69.2 | 70.0 | 72.5 | 64.5 | 5.6 | .53 | .85 | .45 |
| Percent Standard | 10.3 | 12.5 | 5.0 | 17.5 | 5.1 | .17 | .98 | .33 |

^{a}Contrasts: Process = main effect of high-moisture ensiling versus dry-rolling; Hybrid = main effect of hybrid 7219 versus hybrid 6409; Inter = interaction of processing method and hybrid type.
^{b}Marbling score: 4.0 = Slight 0; 4.5 = Slight 50; 5.0 = Small 0, etc.
^{c,d,e}Means within a row bearing unlike superscripts differ (P < .10).

TABLE 3
Effect of corn hybrid and processing method on total tract nutrient
digestibility in finishing steers.

| | 7219 | | 6409 | | | Contrasts^{a} | | |
|---|---|---|---|---|---|---|---|---|
| Item | HMC | DRC | HMC | DRC | SEM | Process | Hybrid | Inter |
| Dry | | | | | | | | |
| Matter | | | | | | | | |
| Intake, | 20.3 | 20.5 | 19.5 | 18.7 | .57 | .66 | .09 | .50 |
| lb/d | | | | | | | | |
| Digest- | 85.0 | 86.1 | 83.3 | 83.8 | 1.0 | .50 | .12 | .80 |
| ibility,% | | | | | | | | |
| Organic | | | | | | | | |
| Matter | | | | | | | | |
| Intake, | 19.4 | 19.6 | 18.7 | 18.0 | .55 | .66 | .10 | .51 |
| lb/d | | | | | | | | |
| Digest | 86.4 | 87.3 | 84.7 | 85.2 | 1.1 | .55 | .15 | .87 |
| ibility, % | | | | | | | | |
| Starch | | | | | | | | |
| Intake, | 12.1 | 11.9 | 10.8 | 11.6 | .36 | .50 | .09 | .24 |
| lb/d | | | | | | | | |
| Digest | 98.1 | 97.6 | 94.9 | 95.5 | 1.0 | .95 | .05 | .61 |
| ibility, % | | | | | | | | |

^{a}Contrasts: Process = main effect of high-moisture ensiling versus dry-rolling; Hybrid = main effect of hybrid 7219 versus hybrid 6409; Inter = interaction of processing method and hybrid type.

TABLE 4
Effect of corn hybrid and processing method on ruminal pH in finishing steers.

| | 7219 | | 6409 | | | Contrasts^{a} | | |
|---|---|---|---|---|---|---|---|---|
| Item | HMC | DRC | HMC | DRC | SEM | Process | Hybrid | Inter |
| Average pH | 5.34 | 5.55 | 5.56 | 5.49 | .22 | .77 | .75 | .59 |
| Maximum pH | 6.12 | 6.07 | 6.44 | 6.15 | .24 | .53 | .46 | .65 |
| Minimum pH | 4.83 | 5.15 | 5.02 | 5.07 | .16 | .34 | .79 | .49 |
| pH change | 1.28 | .92 | 1.42 | 1.09 | .14 | .07 | .34 | .93 |
| pH variance | .102 | .046 | .133 | .049 | .029 | .08 | .62 | .68 |
| Area <5.6^{b} | 507 | 428 | 273 | 310 | 146 | .89 | .31 | .72 |

^{a}Contrasts: Process = main effect of high-moisture ensilling versus dry-rolling; Hybrid = main effect of hybrid 7219 versus hybrid 6409; Inter = interaction of processing method and hybrid type.
^{b}Area <5.6=

TABLE 5
Effect of corn hybrid and processing method on ruminal VFA and ammonia
concentrations in finishing steers.

| | 7219 | | 6409 | | | Contrasts^{a} | | |
|---|---|---|---|---|---|---|---|---|
| Item | HMC | DRC | HMC | DRC | SEM | Process | Hybrid | Inter |
| Total VFA, mM | 75.0 | 77.4 | 75.1 | 75.2 | .74 | .74 | .78 | .76 |
| Acetate, % | 47.3 | 47.5 | 51.6 | 48.1 | 2.6 | .56 | .42 | .53 |
| Propionate, % | 34.4 | 39.6 | 28.4 | 37.8 | 2.3 | .01 | .08 | .30 |
| Acetate:Propionate ratio | 1.41 | 1.22 | 2.00 | 1.31 | .23 | .12 | .21 | .33 |
| Butyrate, % | 10.3 | 7.7 | 11.2 | 7.3 | 1.9 | .13 | .90 | .77 |
| Isobutyrate, % | .83 | .67 | .87 | .96 | .17 | .82 | .27 | .40 |
| Valerate, % | 6.0 | 3.8 | 6.7 | 4.3 | 1.1 | .09 | .65 | .95 |
| Isovalerate, % | 1.02 | .71 | 1.25 | .74 | .23 | .14 | .62 | .71 |
| Ruminal ammonia, mg/dl | 2.90 | 3.46 | 2.57 | 3.17 | .33 | .07 | .28 | .93 |

^{a}Contrasts: Process = main effect of high-moisture ensiling versus dry-rolling Hybrid = main effect of hybrid 7219 versus hybrid 6409; Inter = interaction of processing method and hybrid type.

The present invention includes, as described in Example I, the selection of corn hybrids genetics having a specific endosperm type to enhance feedlot animal performance. The present invention as described above is shown to establish at least an 8.7% improvement in feed efficiency when corn grain from a floury endosperm corn hybrid is used as the grain component of a feed ration.

### EXAMPLE II

### Effects of Endosperm Type and Kernel Processing of Corn Silage on Starch and Fiber Digestibility and Short-Term Lactational Performance of Dairy Cows

Corn Silage Production:

Three corn hybrids are planted, but only two are selected for use in the experiments. Two hybrids (Cargill 6409GQ and Wilson 1698) had a higher content of vitreous endosperm, and one hybrid (NX7219) had greater floury endosperm. The two hybrids chosen for these experiments are Cargill 6409 (vitreous) and NX7219 (floury) because they had the most similar NDF content at harvest (see discussion in next paragraph).

The hybrids are planted in adjacent fields of up to 12 acres each with 16 border rows for each plot to preclude cross-pollination. Maturity of each hybrid is monitored weekly beginning Aug. 15, 1999 and biweekly beginning at early dent stage of maturity. Hybrids are harvested just before physiological maturity as indicted by kernel black layer formation. One-half of each field is harvested with a chopper with rollers set at 1-mm clearance (John Deere, Model 6710 at each location) and the other half unprocessed (not rolled). The unprocessed corn forage is chopped at 0.95 cm (Nebraska) and 1.04 cm (Michigan) theoretical length of cut and the processed (rolled) corn forage is chopped at 1.91 cm (Nebraska) and 2.24 cm (Michigan) theoretical length of cut. Each of the eight corn silage treatments is ensiled in 2.4-m diameter by 30.5-m long plastic silage bags. At both sites, bags are oriented in the same direction with the finished end facing away from prevailing winds. After 45 days of ensiling, each bag is subsampled at 0.6-m intervals and analyzed for dry matter (DM), NDF, crude protein (CP), and fraction of broken kernels and particle size distribution with the Penn State Forage Particle Separator. The NDF content is analyzed using a common methodology between locations in which amylase and sodium sulfite are included (Van Soest et al., 1991). Using these preliminary data, we selected the vitreous endosperm hybrid that had a NDF content closest to the floury hybrid which is Cargill 6409 GQ.

Diets and Treatment Structure:

A 2×2 factorial arrangement of treatments is used: 1) endosperm type (floury or vitreous) and 2) silage processing (rolled or unrolled). The design is a 4×4 Latin square with 28-day periods (21 days for dietary adaptation and 7 days for sample and data collection). At each location, four diets are formulated, each with one of the four corn silage treatments, using the post-ensiled chemical analysis at 45 days. Diets contained approximately 40% corn silage, 10% alfalfa silage, plus corn grain, soybean meal, source(s) of ruminally undegraded protein (RUP), minerals, and vitamins. The specific ingredient and chemical composition of the diets fed at each location are presented in Tables 1 to 4. Approximately equal NDF content is established among the diets; therefore, the percentage of corn silage in the diet varied slightly between the two hybrids. Third cutting alfalfa is harvested at the bud stage and chopped at 0.95-cm theoretical length of cut and ensiled in 2.5-m diameter plastic silage bags. Diets are formulated to contain approximately 28 to 30% NDF and 18.0% CP. Diets are fed as total mixed rations and offered once daily in amounts to ensure 10% feed refusal.

Experimental Animals:

At one site, eight ruminally fistulated, multiparous (BW=649±30 kg) and four intact primiparous (BW=638±27 kg) Holstein dairy cows are assigned to the four diets described in the previous section. At initiation of the experiment, the cows averaged 67±7 days in milk. At a second site, eight ruminally fistulated, multiparous (BW=610±58 kg) Holstein dairy cows are assigned to the four diets. Average days in milk at the start of the trial are 97±29. Cows are housed under conditions described in animal use protocols approved by the Institutional Animal Care and Use Committees at the University of Nebraska and Michigan State University.

Sample and Data Collection:

Cows are weighed and body condition scores are recorded at the beginning of the trial and at the end of each period immediately after the a.m. milking. Body condition scores are evaluated by trained individuals using the 1 (thin) to 5 (obese) scale of Wildman et al. (1982). Milk production is measured daily during the last 7 days of each period. Milk samples are collected at each milking on three days of each period (days 21, 25, and 28 for Nebraska; days 22, 24, and 26 for Michigan) and analyzed for fat, protein, and lactose contents. During the 7-day collection period orts, total mixed ration, and individual ingredients are sampled daily, composited, oven-dried (60° C.), and ground through a Wiley mill (1-mm screen; Arthur H. Thomas Co., Philadelphia, Pa.) for analysis of DM, CP (AOAC, 1990), NDF (Van Soest et al. 1991), ADF (Nebraska only), Van Soest et al., 1991)), 30-h in vitro digestibility (Goering and Van Soest, 1970), and starch (Herrera-Saldana and Huber, 1989) for Nebraska; Oba and Allen (2000) for Michigan). Particle size distributions are determined on fresh samples of the silages and total mixed rations using the Penn State Particle Separator (Lammers et al., 1999). The fraction of kernels that are broken by processing are determined manually. All identifiable pieces of kernel and whole kernels from 200 to 300-g samples of corn silage are removed and the percentage of broken and whole kernels is calculated.

Fecal samples are collected every 9 h for 3 days beginning on day 22 for determination of total tract digestibility of DM, OM, starch, and NDF using 120-h indigestible NDF as an internal marker. Ruminal fluid samples are collected every 4 h (Nebraska) and every 3 h (Michigan) for 24 h on day 26 of each period, immediately analyzed for pH, and frozen for later analysis for volatile fatty acids (VFA) by gas-liquid chromatography (Nebraska) and high-performance liquid chromatography (Michigan). On d 38, ruminal contents of the fistulated cows are removed and weighed to determine ruminal digesta weight and volume. Representative sub-samples are collected for each cow in each period and frozen for later analysis of DM, CP (Nebraska only), OM, NDF, and starch for calculations of pool size and turnover rates (Oba and Allen, 2000). The ruminal pool sizes (kilograms) of DM, CP (Nebraska only), OM, NDF, and starch are determined by multiplying the concentrations of each component by the ruminal digesta DM weight. Turnover rate of digesta in the rumen is calculated by dividing intake of a feed component by ruminal pool size of the component:
Turnover rate in the rumen (%/h)=(intake of component, g/ruminal pool of component, g)/24×100.

All cows are observed every 5 min for a 24-h period on one day per period (d 23 for Nebraska, d 22 for Michigan) for chewing activity. Cows are recorded as ruminating, eating, or neither. From this data, eating and ruminating times per day and per kilogram of NDF intake are calculated, as well as number of meals and rumination bouts.

Statistical Analysis:

The combined data (for both locations) is analyzed as a replicated 4×4 Latin square design with a 2×2 factorial arrangement of the diets and model effects for location, period, square, processing method, endosperm type, and all possible interactions. Statistical analysis is conducted by the use of the Mixed Model procedure of SAS (1998) and the fit-model procedure of JMP (2000). In addition, data for each location is analyzed using the same model with the location effect removed. Discussion of the data focuses on the combined data set except when significant location effects occurred. Significance is declared at P<0.10 unless otherwise noted.

Chemical Composition, Particle Size, and Kernel Integrity:

At one site, the DM content of the silages averaged 42±2%, although the DM content of the nonprocessed, vitreous endosperm corn is greater (P<0.01) than all other corn silages at harvest (Table 1). The NDF, acid detergent fiber (ADF), starch, and CP contents are similar among all silages.

Table 1 shows the effectiveness of processing of the corn silage. Every kernel evaluated contained some degree of damage to the pericarp when the kernel processor is installed. Table 1 also shows the distribution of corn silage particles using the Penn State Particle Separator. There is little difference in particle distribution between the two hybrids when the kernel processor is not installed. When the kernel processor is installed, the percentage of particles left on the top screen is reduced. The vitreous endosperm kernel appeared to shatter and break into smaller pieces during processing, thereby increasing the percentage of corn found in the bottom pan and reducing the amount of particles found on the middle screen. The floury endosperm hybrid left more of the corn silage on the middle screen because of the longer cut and less shattering of the kernel when the kernel processor is installed, thereby reducing the percentage of corn silage found in the bottom pan compared with nonprocessed floury endosperm and the processed vitreous endosperm. Table 2 shows the dietary ingredients and the chemical analysis of each diet. Dry matter content of the nonprocessed, vitreous endosperm diet is greater (P<0.001) than all other diets reflecting the higher DM content of the corn silage. All other nutrient concentrations are similar among diets.

At the second site, the DM content of the silages did not differ and averaged 52.3±1.6% (Table 3). The NDF, ADF, starch, and CP contents are similar among all silages.

Table 3 shows the effectiveness of processing of the corn silage. Nearly all kernels are fractured when the kernel processor is installed. Whole kernels contained over 30% of the total starch for the unprocessed silage but less than 2% of the total starch for the processed silage for each hybrid. Table 3 also shows the distribution of corn silage particles using the Penn State Particle Separator. There are no differences in particle distribution between the two hybrids when the kernel processor is not installed. Processing at a longer TLC affected both hybrids similarly. For each hybrid, processed silage had a greater fraction on the top screen, less on the middle screen and no difference for the bottom pan compared to unprocessed silage. Processing greatly altered the distribution of starch on the screens, shifting the majority of the starch (>60) from the middle screen to the bottom pan for each hybrid.

Table 4 shows the dietary ingredients and the chemical analysis of each diet. Dry matter content of the diets containing the vitreous hybrid (6409) are slightly greater those of the floury hybrid (7219). All other nutrient concentrations are similar among diets.

Nutrient Intake, Milk Production and Composition, and Body Weight:

There is a significant (P<0.01) effect of rolling on DMI, but no effect of endosperm (Table 5). This response is consistent across locations. Milk yield is unaffected by endosperm, but rolling increased (P<0.03) milk yield by 1.6 kg/d. There is a significant (P<0.01) location by rolling interaction; rolling had no effect on milk yield at Michigan State University, but increased milk by 3.3 kg/d at University of Nebraska (Tables 10 and 14). In a review by Johnson et al. (1999), they noted that the reported response to rolling corn silage ranged between 0.2 to 2.0 kg/cow d⁻¹. Production of 4% fat-corrected milk (FCM) and 3.5% solids-corrected milk is unaffected by endosperm or rolling of corn silage. Again, there is a significant (P<0.01) location by rolling interaction; rolling had no effect on FCM production at Michigan, but increased FCM by 2.2 kg/d at Nebraska. The lack of effect of endosperm type on milk and FCM production is consistent across both locations.

Milk fat percentage is increased (P<0.05) for cows fed floury versus vitreous endosperm and is reduced (P<0.06) by silage processing (Table 5). There is a significant (P<0.09) interaction of endosperm and rolling in which milk fat % is reduced by 7% with rolling for the vitreous hybrid, but is unaffected for the floury hybrid. This effect is consistent across both locations (Tables 10 and 14). Percentage of milk protein and lactose are unaffected by diets. Most importantly, the production of milk fat, protein, and lactose are unaffected by diet.

Efficiency of FCM production (FCM/DMI, kg/kg) is increased (P<0.04) by 4.3% for floury versus vitreous corn silage and by 6% (P<0.01) for rolled versus unrolled corn silage (Table 5). Neither body weight nor body condition score change per 28-d period is affected by diet. Similarly, Bal et al. (1998) reported similar body weight gain when lactating dairy cows are fed corn silage harvested at ½ milk line, with or without rolling of the silage.

Fecal Composition and Total Tract Nutrient Digestibility:

Tables 12 and 16 show the composition of feces collected and composited during each period for measurement of total tract digestibility at each location. From these values, and using indigestible NDF as an internal maker, total tract digestibilities are calculated. For both locations, the floury endosperm and rolling resulted in less fecal starch.

Floury endosperm corn silage resulted in a significant increase in digestibility of DM (P<0.02), OM (P<0.01), and starch (P<0.0001), and a concomitant decrease in NDF digestibility (P<0.02; Table 6). This response is consistent for both locations (Tables 11 and 15). Rolling resulted in a significant reduction in digestibility of DM (P<0.05), OM (P<0.05), and NDF (P<0.01), and an increase in starch digestion (P<0.0001). There is no interaction of endosperm by rolling. The effects of endosperm and rolling on total tract digestibility are consistent for both locations. Bal et al. (1998) reported that corn silage processing increased dietary starch digestibility in the total digestive tract of lactating dairy cows from 83.8 to 87.9%.

Papers reporting whole tract digestibilities of two different endosperm types in corn silage could not be found for comparison. However, in two in situ trials, Philippeau and Michalet-Doreau (1997) reported that ruminal starch degradability is 61.3 and 40.1% for dent compared with flint genotypes, respectively. Their second trial results showed that in unensiled, chopped grains, ruminal starch degradability is higher for dent corn than for flint (72.3% vs. 61.6%) corn (Philippeau and Michalet-Doreau, 1998). Mostly attributed to the protein matrix in grain, different types of grains have different starch digestibilities and rates of fermentation. In vitreous endosperm (flint), starch granules are surrounded by protein bodies and are embedded in a dense matrix, which may limit the action of hydrolytic enzymes. In contrast, the starch granules in floury endosperm (dent) are more accessible to hydrolytic enzymes because of a discontinuous protein matrix.

Ruminal pH, Volatile Fatty Acid, and Turnover Rates:

There is no effect of rolling or endosperm on ruminal pH (Table 7). There is a significant (P<0.03) location by endosperm interaction; endosperm type did not affect ruminal pH at Michigan, but the floury endosperm resulted in a small but significant (P<0.04) reduction in ruminal pH at Nebraska (Tables 13 and 17). Similarly, total volatile fatty acid (VFA) concentration, and acetate to propionate ratio are unaffected by treatment at both locations. Ruminal digesta volume (L) is unaffected by endosperm type and rolling (Table 8). However, there is a significant (P<0.06) endosperm by rolling interaction; rolling increased volume for the vitreous hybrid, but decreased volume for the floury corn hybrids (Table 8). The digesta density (kg/L) is unaffected by treatment. Ruminal mass (wet weight) is reduced (P<0.06) for cows fed floury versus vitreous corn silage. There is a significant (P<0.05) endosperm by rolling interaction; processing had no effect on wet weight for the vitreous, but reduced wet weight for the floury hybrid. The ruminal mass of DM, OM, and NDF are unaffected by treatment, but mass of starch is decreased by rolling (P<0.04). Ruminal turnover rates for DM, OM, NDF, and starch are unaffected by treatment (Table 8).

Chewing Behavior:

Eating time (min/kg NDF intake) is unaffected by treatment. However, rumination and total chewing time (min/kg NDF intake) are decreased (P<0.03) for cows fed the vitreous hybrid compared with the floury hybrid. This response may be due to the difference in the percentage of corn silage found on the middle screen (61.2 vs. 67.0%) and in the pan (35.0 vs. 30.2%) of the Penn State Particle Separator for the vitreous endosperm compared with the floury endosperm silage, respectively. Number of meals and ruminating bouts are unaffected by treatment.

TABLE 1
Chemical composition, digestibility; kernel integrity, and particle size of corn
silage treatments at University of Nebraska.

| | | Hybrid¹ | | | | | | | |
|---|---|---|---|---|---|---|---|---|---|
| | | Vitreous | | Floury | | | P< | | |
| Item | | Nonproc | Proc | Nonproc | Proc | SEM | Endo | Proc | E × P |
| 1. | Composition (DM Basis) | | | | | | | | |
| 2.. | DM, % | 48.2 | 43.0 | 39.3 | 39.0 | 2.3 | <0.001 | 0.04 | 0.07 |
| 3. | CP, % | 8.6 | 8.7 | 8.5 | 8.5 | 0.3 | NS² | NS | NS |
| 4. | ADF, % | 23.5 | 24.1 | 22.7 | 22.0 | 1.7 | NS | NS | NS |
| 5. | NDF, % | 47.1 | 45.9 | 42.5 | 43.0 | 2.1 | 0.13 | NS | NS |
| 6. | Starch, % | 31.3 | 30.9 | 30.6 | 32.2 | 2.3 | NS | NS | NS |
| 7. | 30-h in vitro NDF dig. % | 38.1 | 35.6 | 33.0 | 34.3 | 1.3 | 0.03 | 0.03 | 0.04 |
| 8. | Kernel integrity | | | | | | | | |
| 9. | Quantitative | | | | | | | | |
| 10. | % whole | 23.1 | 0.0 | 42.7 | 0.0 | | | | |
| 11. | % fractured | 76.9 | 100.0 | 57.3 | 100.0 | | | | |
| 12. | Calculated³ | | | | | | | | |
| 13. | % whole | 17.6 | 0.0 | 33.8 | 0.0 | | | | |
| 14. | % fractured | 82.4 | 100.0 | 66.2 | 100.0 | | | | |
| 15. | Penn State Particle Size Separator | | | | | | | | |
| 16. | Upper screen, % | 6.0 | 6.7 | 7.9 | 2.8 | | | | |
| 17. | Middle screen, % | 67.8 | 59.3 | 62.6 | 62.7 | | | | |
| 18. | Bottom screen, % | 26.3 | 34.1 | 29.6 | 35.4 | | | | |

¹Hybrids used are Vitreous = Cargill 6409GQ and Floury = Syngenta NX7219.
²Nonsignificant (P > 0.15).
³Grain content is measured. Whole kernels are removed from 250 g of corn silage and by difference cracked kernels are calculated.

TABLE 2
Diet composition and chemical analysis of dietary treatments at University of Nebraska.

| | | Hybrid¹ | | | | | | | |
|---|---|---|---|---|---|---|---|---|---|
| | | Vitreous | | Floury | | | P< | | |
| Item | | Nonproc | Proc | Nonproc | Proc | SEM | Endo | Proc | E × P |
| 1. | Ingredients (% of DM) | | | | | | | | |
| 2. | 6409GQ Nonprocessed | 42.6 | — | — | — | | | | |
| 3. | 6409GQ Processed | — | 42.6 | — | — | | | | |
| 4. | NX7219 Nonprocessed | — | — | 45.9 | — | | | | |
| 5. | NX7219 Processed | — | — | — | 45.9 | | | | |
| 6. | Alfalfa haylage | 10.0 | 10.0 | 10.0 | 10.0 | | | | |
| 7. | Corn grain | 23.0 | 23.0 | 19.9 | 19.9 | | | | |
| 8. | Soybean meal | 17.2 | 17.2 | 17.2 | 17.2 | | | | |
| 9. | SoyPass ®² | 3.0 | 3.0 | 2.8 | 2.8 | | | | |
| 10. | Blood meal | 0.7 | 0.7 | 0.7 | 0.7 | | | | |
| 11. | Base mix³ | 3.5 | 3.5 | 3.5 | 3.5 | | | | |
| 12. | Composition (DM Basis) | | | | | | | | |
| 13. | DM, % | 60.7 | 56.5 | 53.8 | 55.2 | 1.2 | <0.001 | 0.03 | <0.001 |
| 14. | CP, % | 18.3 | 17.8 | 17.7 | 17.9 | 1.0 | NS⁵ | NS | NS |
| 15. | RUP, %⁴ | 6.6 | 6.6 | 6.5 | 6.5 | | | | |
| 16. | ADF, % | 15.7 | 15.5 | 15.0 | 15.8 | 0.6 | NS | NS | NS |
| 17. | NDF, % | 30.8 | 32.5 | 31.7 | 32.0 | 1.6 | NS | NS | NS |
| 18. | Starch, % | 35.5 | 34.5 | 36.0 | 35.9 | 1.8 | NS | NS | NS |
| 19. | OM, % | 91.1 | 91.4 | 91.6 | 91.4 | 0.4 | NS | NS | NS |
| 20. | NE_{I}, Mcal/kg⁴ | 1.69 | 1.69 | 1.69 | 1.69 | | | | |

¹Hybrids used are Vitreous = Cargill 6409GQ, and Syngenta NX7219.
²Nonenzymatically browned soybean meal (Lignotech USA, Rothschild, WI).
³Base mix 23.8% Ca, 8.4% P, 14.4% K, 7.1% Mg, 3.4% S, 10.7% Na, 16.6% CI, 2,550 mg/kg Zn, 6,771 mg/kg Fe, 1,756 mg/kg Mn, 319 mg/kg Cu, 42 mg/kg I, 23 mg/kg Co, 10 mg/kg Se, 78.9 KIU/kg, 15.8 KIU/kg, and 603 IU/kg.
⁴Estimated using values for individual ingredients given by NRC (1989).
⁵Nonsignificant (P > 0.15).

TABLE 3
Chemical composition, digestibility, kernel integrity, and particle
size of corn silage treatments at Michigan State University.

| | Hybrid¹ | | | |
|---|---|---|---|---|
| | Vitreous | | Floury | |
| Item | nonproc | proc | nonproc | proc |
| DM % | 52.3 | 53.3 | 50.6 | 52.8 |
| | % of DM | | | |
| NDF | 41.4 | 41.9 | 39.8 | 41.2 |
| CP | 7.0 | 7.2 | 6.7 | 7.5 |
| Ash | 2.5 | 2.5 | 2.4 | 2.5 |
| Starch | 36.6 | 35.5 | 36.9 | 34.6 |
| Particle Size (% of DM) | | | | |
| Top | 9.85^{b} | 11.78^{a} | 9.68^{b} | 12.68^{a} |
| Middle | 57.15^{a} | 53.28^{b} | 57.13^{a} | 53.73^{b} |
| Bottom | 32.98 | 34.95 | 33.18 | 33.63 |
| Starch distribution (% of Starch) | | | | |
| Top | 0.80^{d} | 1.55^{b} | 1.07^{c} | 2.05^{a} |
| Middle | 65.7^{a} | 30.9^{b} | 65.5^{a} | 37.7^{b} |
| Bottom | 33.5^{b} | 67.6^{a} | 33.4^{b} | 60.3^{a} |
| Corn Silage Distribution | | | | |
| (% of DM) | | | | |
| Whole kernels | 22.3^{a} | 0.6^{c} | 17.2^{b} | 0.3^{d} |
| Fractured kernels | 26.5^{b} | 42.1^{a} | 27.8^{b} | 39.2^{a} |
| >2360 μm | 40.3^{b} | 43.1^{b} | 48.6^{a} | 47.5^{a} |
| <2360 μm | 10.9^{b} | 14.3^{a} | 6.4^{b} | 13.0^{a} |
| Starch distribution (% of Starch) | | | | |
| Whole kernels | 39.0^{a} | 1.2^{b} | 31.7^{a} | 0.5^{b} |
| Fractured kernels | 48.0^{b} | 75.6^{a} | 54.0^{b} | 72.8^{a} |
| >2360 μm | 5.6 | 7.5 | 8.2 | 9.6 |
| <2360 μm | 7.5^{b} | 15.7^{a} | 6.1^{b} | 17.0^{a} |
| Ph | 4.18 | 4.10 | 4.03 | 4.13 |

Means for nutrient composition in same row followed by different superscript letters differ significantly (P < 0.05).
¹Hybrids used are Vitreous = Cargill 6409GQ and Floury = Syngenta NX7219.
²Interaction between hybrid and process is significant. (P = .03)

TABLE 4
Ingredients and chemical composition of experimental diets
(% of dietary DM) at Michigan State University.

| | Hybrid¹ | | | |
|---|---|---|---|---|
| | Vitreous | | Floury | |
| Item | nonproc | proc | nonproc | proc |
| Dietary ingredients | | | | |
| Corn silage | 32.0 | 32.5 | 33.7 | 35.1 |
| Alfalfa silage | 11.4 | 11.3 | 11.5 | 11.3 |
| Dry ground shelled corn | 28.5 | 28.3 | 26.3 | 25.8 |
| Protein mix | 24.0 | 23.9 | 24.3 | 23.8 |
| Vitamin and mineral mix | 4.1 | 4.1 | 4.1 | 4.1 |
| Nutrient composition | | | | |
| DM | 69.0^{a} | 69.4^{a} | 67.3^{b} | 68.2^{b} |
| NDF | 24.3 | 24.6 | 24.4 | 25.2 |
| CP | 19.4 | 19.5 | 19.4 | 19.4 |
| Starch | 35.8 | 35.4 | 35.1 | 34.3 |
| Corn silage NDF (% of dietary DM) | 13.2 | 13.6 | 13.4 | 14.4 |
| Corn silage NDF (% of dietary NDF) | 54.3 | 55.2 | 55.0 | 57.3 |
| Corn silage starch (% of dietary DM) | 11.7 | 11.5 | 12.5 | 12.2 |
| Corn silage starch (% of dietary starch) | 32.7 | 32.5 | 35.5 | 35.4 |

¹Hybrids used are Vitreous = Cargill 6409GQ and Floury = Syngenta NX7219.
Means for nutrient composition in same row followed by different superscript letters differ significantly (P < 0.05).

TABLE 5
Effects of processing and corn hybrid on DMI and milk yield (combined data set).

| | Hybrid¹ | | | | | | | | |
|---|---|---|---|---|---|---|---|---|---|
| | Vitreous | | Floury | | | P value | | | |
| Item | nonproc | proc | nonproc | proc | SE | Endo | Proc | E × P | Loc Int |
| Intake | | | | | | | | | |
| DMI (kg/d) | 27.4 | 27.0 | 27.6 | 26.1 | 0.51 | NS | .01 | .12 | NS |
| Yield (kg/d) | | | | | | | | | |
| Raw milk | 40.9 | 43.1 | 41.8 | 42.7 | 0.95 | NS | .03 | NS | B = .01 |
| FCM (4%) | 38.5 | 39.2 | 39.5 | 40.1 | 1.00 | NS | NS | NS | B = .01 |
| SCM | 40.7 | 40.3 | 40.9 | 40.6 | 0.83 | NS | NS | NS | NS |
| Milk fat | 1.56 | 1.50 | 1.56 | 1.55 | 0.04 | NS | NS | NS | NS |
| Milk protein | 1.21 | 1.22 | 1.24 | 1.21 | 0.02 | NS | NS | NS | B = .16 |
| Milk lactose | 2.04 | 2.09 | 2.06 | 2.06 | 0.04 | NS | NS | NS | NS |
| Milk composition (%) | | | | | | | | | |
| Fat | 3.68 | 3.44 | 3.69 | 3.68 | 0.09 | .05 | .06 | .09 | NS |
| Protein | 2.84 | 2.86 | 2.90 | 2.86 | 0.03 | NS | NS | .13 | NS |
| Lactose | 4.74 | 4.77 | 4.78 | 4.78 | 0.04 | NS | NS | NS | NS |
| FCM/DMI | 1.40 | 1.45 | 1.43 | 1.55 | 0.04 | .04 | <.01 | NS | B = .05 |
| BW change (kg/d) | 0.015 | 0.016 | 0.017 | 0.007 | 0.006 | NS | NS | NS | NS |
| BCS change (/28 d) | 0.14 | 0.13 | 0.10 | 0.09 | 0.06 | NS | NS | NS | C = .08 |

¹Hybrids used are Vitreous = Cargill 6409GQ and Floury = Syngenta NX7219.
A: location × Endo
B: location × proc
C: location × endo × proc

TABLE 6
Effects of processing and corn hybrid on digestibilities
of DM, OM, NDF and starch (combined data set).

| | Hybrid¹ | | | | | | | | |
|---|---|---|---|---|---|---|---|---|---|
| | Vitreous | | Floury | | | P value | | | |
| Item | nonproc | proc | nonproc | proc | SE | Endo | Proc | E × P | Loc Int |
| DM | | | | | | | | | |
| Intake (kg/d) | 27.4 | 27.0 | 27.6 | 26.1 | 0.51 | NS | .01 | .12 | NS |
| Apparently digested in total tract | | | | | | | | | |
| (kg/d) | 17.3 | 16.6 | 18.3 | 16.6 | 0.54 | .15 | <.01 | .17 | B = .04 |
| (%) | 66.2 | 63.3 | 68.8 | 66.8 | 1.59 | .02 | .05 | NS | B = .002 |
| OM | | | | | | | | | |
| Intake (kg/d) | 25.3 | 24.9 | 25.6 | 24.1 | 0.47 | NS | .01 | .10 | NS |
| Apparently digested in total tract | | | | | | | | | |
| (kg/d) | 16.6 | 15.8 | 17.5 | 16.1 | 0.40 | .04 | <.01 | NS | B = .03 |
| (%) | 68.6 | 65.1 | 71.2 | 69.9 | 1.56 | <.01 | .05 | NS | A = .14, |
| | | | | | | | | | B = <.01 |
| NDF | | | | | | | | | |
| Intake (kg/d) | 7.4 | 7.5 | 7.5 | 7.5 | 0.24 | NS | NS | NS | NS |
| Apparently digested in total tract | | | | | | | | | |
| (kg/d) | 3.2 | 2.9 | 3.3 | 3.0 | 0.13 | NS | <.01 | NS | NS |
| (%) | 45.4 | 40.1 | 46.2 | 43.1 | 1.43 | .09 | <.01 | NS | .03 |
| Starch | | | | | | | | | |
| Intake (kg/d) | 9.96 | 10.0 | 9.98 | 9.20 | 0.24 | .03 | .05 | .02 | NS |
| Apparently digested in total tract | | | | | | | | | |
| (kg/d) | 8.07 | 8.82 | 8.54 | 8.36 | 0.22 | NS | .09 | <.01 | B = .08 |
| (%) | 83.7 | 91.4 | 88.2 | 94.6 | 1.07 | <.0001 | <.0001 | NS | A = .04 |
| CP | | | | | | | | | |
| Intake (kg/d) | 4.61 | 4.61 | 4.59 | 4.56 | 0.09 | NS | NS | NS | NS |
| Apparently digested in total tract | | | | | | | | | |
| (kg/d) | 3.00 | 2.87 | 3.06 | 2.95 | 0.12 | NS | .14 | NS | B = .06 |
| (%) | 65.7 | 62.4 | 67.1 | 65.2 | 1.65 | .10 | .04 | NS | B = .01 |

¹Hybrids used are Vitreous = Cargill 6409GQ and Floury = Syngenta NXJ219.
A: location × endo
B: location × proc
C: location × endo

TABLE 7
Effects of processing and corn hybrid on ruminal fermentation (combined data set).

| | Hybrid¹ | | | | | | | | |
|---|---|---|---|---|---|---|---|---|---|
| | Vitreous | | Floury | | | P value | | | |
| Item | nonporc | proc | nonproc | proc | SE | Endo | Proc | E × P | Loc Int |
| Ruminal pH | | | | | | | | | |
| Mean | 5.99 | 6.01 | 6.00 | 6.01 | 0.04 | NS | NS | NS | A = .03 |
| Maximum | 6.41 | 6.55 | 6.54 | 6.59 | 0.08 | .17 | .13 | NS | NS |
| Minimum | 5.86 | 5.58 | 5.56 | 5.61 | 0.04 | NS | NS | NS | NS |
| Range | 1.13 | .67 | 1.12 | 0.83 | 0.09 | .14 | .16 | .18 | B = .14 |
| Total VFA (mM) | 111.1 | 107.1 | 108.7 | 106.9 | 2.76 | NS | NS | NS | NS |
| VFA (mol/100 mol) | | | | | | | | | |
| Acetate | 54.6 | 53.2 | 53.3 | 53.7 | 1.22 | NS | NS | NS | NS |
| Propionate | 20.1 | 20.8 | 19.5 | 20.9 | 0.91 | NS | .14 | NS | NS |
| Butyrate | 10.4 | 9.6 | 10.7 | 10.2 | 0.45 | NS | .09 | NS | C = .05 |
| Valerate | 1.49 | 1.60 | 1.48 | 1.59 | 0.11 | NS | .19 | NS | NS |
| Branched-chain VFA | 2.34 | 2.26 | 2.26 | 2.32 | 0.09 | NS | NS | NS | NS |
| Acetate:Propionate ratio | 2.82 | 2.70 | 2.79 | 2.66 | 0.11 | NS | .16 | NS | NS |

¹Hybrids used are Vitreous = Cargill 6409GQ and Floury = Syngenta NX7219.
A: location × endo,
B: location × proc,
C: location × endo × proc

TABLE 8
Effects of processing and corn hybrid on digesta characteristics and digestion
kinetics in the rumen (combined data set).

| | Hybrid¹ | | | | | | | | |
|---|---|---|---|---|---|---|---|---|---|
| | Vitreous | | Floury | | | P value | | | |
| Item | nonproc | proc | nonproc | proc | SE | Endo | Proc | E × P | Loc Int |
| Rumen digesta volume (L) | 98.2 | 101.7 | 100.3 | 95.4 | 2.81 | NS | NS | .06 | C = .05 |
| Density (kg/L) | 0.83 | 0.80 | 0.81 | 0.80 | 0.01 | NS | NS | NS | NS |
| Rumen mass (kg) | | | | | | | | | |
| Wet digesta | 80.4 | 81.4 | 80.5 | 76.2 | 1.72 | .06 | NS | .05 | C = .01 |
| DM | 14.6 | 14.5 | 14.6 | 13.7 | 0.48 | NS | .18 | .19 | NS |
| OM | 13.1 | 13.1 | 13.3 | 12.1 | 0.47 | NS | .10 | .15 | C = .12 |
| NDF | 8.6 | 8.0 | 7.9 | 7.4 | 0.51 | .11 | .17 | NS | C = .13 |
| Starch | 0.92 | 0.82 | 0.98 | 0.72 | 0.12 | NS | .04 | NS | NS |
| Ruminal turnover rate (%/h) | | | | | | | | | |
| DM | 7.89 | 7.60 | 7.74 | 7.77 | 0.34 | NS | NS | NS | A = .14, C = .15 |
| OM | 8.01 | 7.76 | 7.90 | 8.07 | 0.36 | NS | NS | NS | C = .08 |
| NDF | 3.86 | 3.92 | 3.95 | 4.15 | 0.21 | NS | .10 | NS | C = .10 |
| Starch | 51.83 | 55.08 | 50.46 | 54.36 | 5.18 | NS | NS | NS | NS |

¹Hybrids used are Vitreous = Cargill 6409GQ and Floury = Syngenta NX7219.
A: location × endo.
B: location × proc,
C: location × endo × proc

TABLE 9
Effects of processing and corn hybrid on chewing behavior (combined data set).

| | Hybrid¹ | | | | | | | | |
|---|---|---|---|---|---|---|---|---|---|
| | Vitreous | | Floury | | | P value | | | |
| Item | nonproc | proc | nonproc | proc | SE | Endo | Proc | E × P | Loc Int |
| Eating time (min) | | | | | | | | | |
| /d | 224 | 214 | 236 | 224 | 8.7 | .09 | .09 | NS | C = .09 |
| /kg of DMI | 8.39 | 8.04 | 8.65 | 8.84 | 0.38 | .07 | NS | NS | B = .16, |
| | | | | | | | | | C = .04 |
| /kg of NDF intake | 31.93 | 29.24 | 32.23 | 31.96 | 1.46 | .17 | .18 | NS | B = .10, |
| | | | | | | | | | C = .05 |
| Ruminating time (min) | | | | | | | | | |
| /d | 457 | 473 | 495 | 497 | 21.0 | .05 | NS | NS | B = .09 |
| /kg of DMI | 16.44 | 17.63 | 17.95 | 19.50 | 0.82 | <.01 | .03 | NS | B = .04 |
| /kg of NDF intake | 62.45 | 64.90 | 67.64 | 70.06 | 3.02 | .03 | NS | NS | B = .007 |
| Total chewing time (min) | | | | | | | | | |
| /d | 681 | 688 | 731 | 721 | 21.5 | .01 | NS | NS | B = .07 |
| /kg of DMI | 24.80 | 25.72 | 26.63 | 28.29 | 0.85 | <.01 | .05 | NS | B = .01 |
| /kg of NDF intake | 94.24 | 94.34 | 99.96 | 101.82 | 3.38 | .01 | NS | NS | B = .002 |
| Meal number (/d) | 7.02 | 6.89 | 7.06 | 6.95 | 0.34 | NS | NS | NS | B = .06, |
| | | | | | | | | | C = .05 |
| Ruminating bouts (/d) | 11.89 | 12.25 | 12.34 | 12.14 | 0.49 | NS | NS | NS | NS |

¹Hybrids used are Vitreous = Cargill 6409GQ and Floury = Syngenta NX7219.
A: location × endo
B: location × proc
C: location × endo × proc

TABLE 10
Lactational performance of cows fed corn silage with two different endosperm types
and processing methods at University of Nebraska.

| | Hybrid¹ | | | | | | | |
|---|---|---|---|---|---|---|---|---|
| | Vitreous | | Floury | | | P< | | |
| Item | Nonproc | Proc | Nonproc | Proc | SEM | Endo | Proc | E × P |
| 1. BW, kg | 646 | 644 | 651 | 641 | 17 | NS² | NS | NS |
| 2. BW change, kg/period | 4.5 | 9.7 | 7.4 | −4.3 | 6.8 | NS | NS | 0.13 |
| 3. BCS | 3.2 | 3.1 | 3.2 | 3.2 | 0.1 | 0.12 | NS | NS |
| 4. BCS change/period | 0.09 | −0.04 | 0.02 | 0.03 | 0.08 | NS | NS | NS |
| 5. DMI, kg | 26.5 | 26.3 | 26.3 | 25.3 | 1.2 | NS | NS | NS |
| 6. DMI, % of BW | 4.1 | 4.1 | 4.0 | 4.0 | 0.2 | NS | NS | NS |
| 7. Milk, kg/d | 34.0 | 37.8 | 34.7 | 37.7 | 2.4 | NS | 0.01 | NS |
| 8. 4% FCM, kg/d | 32.4 | 34.9 | 33.0 | 35.9 | 2.5 | NS | 0.04 | NS |
| 9. Efficiency, FCM/DMI | 1.22 | 1.33 | 1.25 | 1.43 | 0.06 | 0.09 | <0.001 | NS |
| 10. Milk fat | | | | | | | | |
| 11. % | 3.73 | 3.53 | 3.73 | 3.72 | 0.17 | 0.12 | 0.08 | 0.11 |
| 12. kg/d | 1.3 | 1.3 | 1.3 | 1.3 | 0.1 | NS | NS | NS |
| 13. Milk true protein | | | | | | | | |
| 14. % | 2.86 | 2.85 | 2.90 | 2.88 | 0.07 | 0.08 | NS | NS |
| 15. kg/d | 1.0 | 1.0 | 1.0 | 1.0 | 0.1 | NS | NS | NS |
| 16. Milk lactose | | | | | | | | |
| 17. % | 4.71 | 4.73 | 4.71 | 4.75 | 0.07 | NS | 0.14 | NS |
| 18. kg/d | 1.7 | 1.7 | 1.7 | 1.7 | 0.1 | NS | NS | NS |

¹Hybrids used are Vitreous = Cargill 6409GQ, and Floury = Syngenta NX7219.
²Nonsignificant (P > 0.15).

TABLE 11
Whole tract digestibilities, eating times, and rumen turnover rates from corn-silage
based diets with two different endosperm types and processing methods
at University of Nebraska.

| | Hybrid¹ | | | | | | | |
|---|---|---|---|---|---|---|---|---|
| | Vitreous | | Floury | | | P< | | |
| Item | Nonproc | Proc | Nonproc | Proc | SEM | Endo | Proc | E × P |
| 1. Total Tract Digestibility | | | | | | | | |
| 2. DM, % | 65.3 | 58.8 | 69.2 | 64.2 | 2.9 | 0.09 | 0.04 | NS² |
| 3. NDF, % | 51.3 | 42.1 | 51.4 | 47.4 | 2.8 | NS | 0.005 | NS |
| 4. OM, % | 67.8 | 59.8 | 71.3 | 67.9 | 3.0 | 0.03 | 0.03 | NS |
| 5. CP, % | 67.9 | 60.6 | 70.2 | 66.1 | 2.4 | 0.13 | 0.04 | NS |
| 6. Starch, % | 82.9 | 92.5 | 89.7 | 97.0 | 0.9 | <0.001 | <0.001 | NS |
| 7. Eating | | | | | | | | |
| 8. min/d | 177 | 174 | 194 | 173 | 19 | NS | NS | NS |
| 9. min/kg of NDF intake | 22.7 | 20.3 | 24.7 | 20.7 | 3.2 | NS | 0.06 | NS |
| 10. Ruminating | | | | | | | | |
| 11. min/d | 444 | 432 | 475 | 444 | 36 | NS | NS | NS |
| 12. min/kg of NDF intake | 54.2 | 50.6 | 58.5 | 53.4 | 4.7 | NS | 0.13 | NS |
| 13. Total chewing | | | | | | | | |
| 14. min/d | 622 | 605 | 673 | 617 | 42 | 0.11 | 0.08 | NS |
| 15. min/kg of NDF intake | 76.9 | 70.8 | 83.2 | 74.2 | 6.7 | 0.14 | 0.03 | NS |
| 16. Rumen Turnover Rate | | | | | | | | |
| 17. DM, %/h | 8.72 | 7.87 | 7.63 | 8.05 | 0.74 | NS | NS | NS |
| 18. NDF, %/h | 4.86 | 4.72 | 4.43 | 5.09 | 0.52 | NS | NS | NS |
| 19. OM, %/h | 9.03 | 8.19 | 7.90 | 8.67 | 0.75 | NS | NS | 0.11 |
| 20. Starch, %/h | 58.4 | 65.5 | 55.1 | 67.6 | 9.3 | NS | NS | NS |

¹Hybrids used are Vitreous = Cargill 6409GQ, and Floury = Syngenta NX7219.
²Nonsignificant (P > 0.15).

TABLE 12
Fecal composition and chemical analysis at University of Nebraska.

| | Hybrid¹ | | | | | | | |
|---|---|---|---|---|---|---|---|---|
| | Vitreous | | Floury | | | P< | | |
| Item | Nonproc | Proc | Nonproc | Proc | SEM | Endo | Proc | E × P |
| 1. Composition (DM Basis) | | | | | | | | |
| 2. DM, % | 16.7 | 15.6 | 16.5 | 14.6 | 0.7 | NS¹ | 0.03 | NS |
| 3. OM, % | 85.1 | 82.7 | 84.4 | 81.9 | 0.9 | NS | 0.001 | NS |
| 4. CP, % | 16.6 | 17.2 | 17.0 | 17.7 | 0.3 | 0.07 | 0.01 | NS |
| 5. NDF, % | 44.7 | 49.6 | 48.2 | 53.7 | 1.4 | 0.002 | <0.001 | NS |
| 6. Starch, % | 15.6 | 7.0 | 10.4 | 3.5 | 0.9 | <0.001 | <0.001 | NS |

¹Hybrids used are Vitreous = Cargill 6409GQ, and Floury = Syngenta NX7219.
⁵Nonsignificant (P > 0.15).

TABLE 13
Ruminal pH and VFA profiles as influenced by corn-silage based diets with two
different endosperm types and processing methods at University of Nebraska.

| | Hybrid¹ | | | | | | | |
|---|---|---|---|---|---|---|---|---|
| | Vitreous | | Floury | | | P< | | |
| Item | Nonproc | Proc | Nonproc | Proc | SEM | Endo | Proc | E × P |
| 1. Ruminal pH | 5.99 | 6.01 | 5.95 | 5.90 | 0.06 | 0.04 | NS² | NS |
| 2. Volatile fatty acids | | | | | | | | |
| 3. Total VFA, mM | 84.8 | 81.3 | 83.1 | 86.9 | 3.5 | NS | NS | NS |
| 4. Acetate, mM | 54.8 | 50.9 | 51.5 | 53.5 | 2.5 | NS | NS | 0.10 |
| 5. Propionate, mM | 17.9 | 18.9 | 17.4 | 19.6 | 1.4 | NS | NS | NS |
| 6. n-Butyrate, mM | 9.1 | 8.1 | 9.3 | 9.8 | 1.0 | NS | NS | NS |
| 7. Iso-butyrate, mM | 0.9 | 0.8 | 0.8 | 0.8 | 0.1 | NS | NS | 0.12 |
| 8. n-Valerate, mM | 1.2 | 1.4 | 1.2 | 1.5 | 0.2 | NS | NS | NS |
| 9. Iso-valerate, mM | 1.5 | 1.3 | 1.4 | 1.4 | 0.1 | NS | NS | NS |
| 10. Acetate:propionate | 3.1 | 2.9 | 3.0 | 2.8 | 0.2 | NS | 0.11 | NS |
| 11. Acetate + butyrate:propionate | 8.2 | 8.8 | 7.8 | 8.2 | 0.7 | NS | NS | NS |

¹Hybrids used are Vitreous = Cargill 6409GQ, and Floury = Syngenta NX7219.
²Nonsignificant (P > 0.15).

TABLE 14
Effects of processing and corn hybrid on DMI and milk yield at Michigan State University.

| | Hybrid¹ | | | | | | | |
|---|---|---|---|---|---|---|---|---|
| | Vitreous | | Floury | | | P value | | |
| Item | nonproc | proc | nonproc | proc | SE | Endo | Proc | Interaction |
| Intake | | | | | | | | |
| DMI (kg/d) | 25.2 | 24.8 | 26.1 | 24.0 | 0.40 | NS | <.01 | .06 |
| Digested DMI (kg/d) | 15.9 | 16.2 | 17.1 | 16.0 | 0.27 | .10 | .13 | <.05 |
| Yield (kg/d) | | | | | | | | |
| Raw milk | 38.3 | 38.8 | 39.5 | 38.2 | 0.82 | NS | NS | NS |
| FCM (4%) | 34.4 | 33.5 | 36.0 | 34.5 | 0.86 | .15 | .19 | NS |
| SCM | 34.1 | 33.6 | 35.8 | 34.2 | 0.91 | NS | NS | NS |
| Milk fat | 1.3 | 1.2 | 1.3 | 1.3 | 0.05 | .18 | NS | NS |
| Milk protein | 1.1 | 1.1 | 1.2 | 1.1 | 0.03 | NS | NS | NS |
| Milk lactose | 1.8 | 1.9 | 1.9 | 1.8 | 0.05 | NS | NS | NS |
| Milk composition (%) | | | | | | | | |
| Protein | 2.86 | 2.93 | 2.94 | 2.88 | 0.05 | NS | NS | NS |
| Fat | 3.36 | 3.11 | 3.41 | 3.41 | 0.14 | NS | NS | NS |
| Lactose | 4.74 | 4.76 | 4.83 | 4.78 | 0.08 | NS | NS | NS |
| FCM/DMI | 1.36 | 1.35 | 1.38 | 1.46 | 0.04 | .14 | NS | NS |
| BW change (kg/d) | 0.008 | −0.128 | −0.022 | −0.112 | 0.118 | NS | NS | NS |
| BCS change (/28 d) | −0.05 | 0.07 | −0.05 | −0.09 | 0.05 | .17 | NS | .15 |

¹Hybrids used arc Vitreous = Cargill 6409GQ and Floury = Syngenta NX7219.

TABLE 15
Effects of processing and corn hybrid on digestibilities of DM, OM, NDF and starch
at Michigan State University.

| | Hybrid¹ | | | | | | | |
|---|---|---|---|---|---|---|---|---|
| | Vitreous | | Floury | | | P value | | |
| Item | nonproc | proc | nonproc | Proc | SE | Endo | Proc | Interaction |
| DM | | | | | | | | |
| Intake (kg/d) | 25.2 | 24.8 | 26.1 | 24.0 | 0.40 | NS | <.01 | .06 |
| Apparently digested in total tract | | | | | | | | |
| (kg/d) | 15.9 | 16.2 | 17.1 | 16.0 | 0.27 | .10 | .13 | <.05 |
| (%) | 63.2 | 65.1 | 65.5 | 66.8 | 0.94 | .05 | .10 | NS |
| OM | | | | | | | | |
| Intake (kg/d) | 23.8 | 23.4 | 24.6 | 22.6 | 0.38 | NS | <.01 | .06 |
| Apparently digested in total tract | | | | | | | | |
| (kg/d) | 15.5 | 15.7 | 16.6 | 15.5 | 0.27 | .13 | .14 | <.05 |
| (%) | 65.2 | 67.1 | 67.4 | 68.8 | 1.01 | .08 | .13 | NS |
| NDF | | | | | | | | |
| Intake (kg/d) | 6.2 | 6.1 | 6.4 | 6.0 | 0.11 | NS | .12 | NS |
| Apparently digested in total tract | | | | | | | | |
| (kg/d) | 2.5 | 2.4 | 2.7 | 2.4 | 0.09 | .17 | <.05 | NS |
| (%) | 40.8 | 39.3 | 42.8 | 41.0 | 1.35 | .19 | NS | NS |
| Starch | | | | | | | | |
| Intake (kg/d) | 9.0 | 8.8 | 9.2 | 8.2 | 0.22 | NS | <.05 | .14 |
| Apparently digested in total tract | | | | | | | | |
| (kg/d) | 7.6 | 8.0 | 8.0 | 7.6 | 0.19 | NS | NS | .09 |
| (%) | 84.5 | 90.9 | 86.9 | 92.8 | 1.09 | .08 | <.001 | NS |
| CP | | | | | | | | |
| Intake (kg/d) | 4.9 | 4.9 | 4.9 | 4.9 | 0.02 | NS | NS | <.01 |
| Apparently digested in total tract | | | | | | | | |
| (kg/d) | 3.1 | 3.3 | 3.3 | 3.3 | 0.06 | NS | NS | NS |
| (%) | 65.5 | 66.7 | 67.2 | 67.2 | 1.10 | NS | NS | NS |

¹Hybrids used are Vitreous = Cargill 6409GQ and Floury = Syngenta NX7219.

TABLE 16
Effects of proccessing and corn hybrid on focal composition at Michigan
State University.

| | Hybrid¹ | | | | | | | |
|---|---|---|---|---|---|---|---|---|
| | Vitreous | | Floury | | | P value | | |
| Item | nonproc | proc | nonproc | Proc | SE | Endo | Proc | Interaction |
| Focal Nutrient Composition (%) | | | | | | | | |
| DM | 16.9 | 15.5 | 16.2 | 15.4 | 0.20 | .06 | <.001 | .12 |
| NDF | 39.3 | 43.0 | 40.3 | 44.9 | 0.56 | <.05 | <.001 | NS |
| CP | 18.2 | 18.5 | 18.3 | 19.2 | 0.30 | .18 | .08 | NS |
| Ash | 11.0 | 11.1 | 11.0 | 11.2 | 0.42 | NS | NS | NS |
| Starch | 14.6 | 9.2 | 13.1 | 7.4 | 0.81 | .07 | <.001 | NS |
| Ether extract | 3.24 | 3.47 | 3.38 | 2.96 | 0.20 | NS | NS | .12 |
| In vitro NDF digestibility | 31.1 | 33.5 | 31.4 | 35.2 | 0.65 | .18 | <.001 | NS |
| Indigestible NDF (as % of NDF) | 61.4 | 59.5 | 61.0 | 59.0 | 1.47 | NS | NS | NS |

¹Hybrids used are Vitreous = Cargill 6409GQ and Floury = Syngenta NX7219.

TABLE 17
Effects of processing and corn hybrid on ruminal fermentation at
Michigan State Univeisity.

| | Hybrid¹ | | | | | | | |
|---|---|---|---|---|---|---|---|---|
| | Vitreous | | Floury | | | P value | | |
| Item | nonproc | proc | nonproc | Proc | SE | Endo | Proc | Interaction |
| Ruminal pH | | | | | | | | |
| Mean | 5.92 | 5.95 | 5.97 | 6.03 | 0.03 | .06 | NS | NS |
| Maximum | 6.52 | 6.57 | 6.68 | 6.72 | 0.08 | .09 | NS | NS |
| Minimum | 5.51 | 5.53 | 5.50 | 5.60 | 0.04 | NS | .18 | NS |
| Range | 1.01 | 1.05 | 1.18 | 1.13 | 0.07 | .12 | NS | NS |
| Total VFA (mM) | 148.0 | 143.8 | 146.3 | 141.2 | 2.8 | NS | .12 | NS |
| VFA (mol/100 mol) | | | | | | | | |
| Acetate | 56.7 | 56.5 | 56.8 | 56.6 | 0.66 | NS | NS | NS |
| Propionate | 26.9 | 27.3 | 25.8 | 27.3 | 0.80 | NS | NS | NS |
| Butyrate | 11.9 | 11.5 | 13.0 | 11.5 | 0.25 | .05 | <.01 | <.05 |
| Valerate | 1.95 | 2.06 | 1.93 | 1.97 | 0.11 | NS | NS | NS |
| Branched-chain VFA | 2.52 | 2.56 | 2.53 | 2.61 | 0.08 | NS | NS | NS |
| Acetate:Propionate ratio | 2.15 | 2.15 | 2.24 | 2.12 | 0.09 | NS | NS | NS |

¹Hybrids used are Vitreous = Cargill 6409GQ and Floury = Syngenta NX7219.

TABLE 18
Effects of processing and corn hybrid on digesta characteristics and digestion
kinetics in the rumen at Michigan State University.

| | Hybrid¹ | | | | | | | |
|---|---|---|---|---|---|---|---|---|
| | Vitreous | | Floury | | | P value | | |
| Item | nonproc | proc | nonproc | Proc | SE | Endo | Proc | Interaction |
| Rumen digesta volume (L) | 96.5 | 95.4 | 94.8 | 93.8 | 2.37 | NS | NS | NS |
| Density (kg/L) | .84 | .82 | .81 | .81 | 0.01 | .14 | NS | NS |
| Rumen mass (kg) | | | | | | | | |
| Wet digesta | 80.7 | 78.2 | 76.8 | 76.2 | 1.8 | .13 | NS | NS |
| DM | 14.2 | 13.7 | 13.6 | 12.9 | 0.59 | NS | NS | NS |
| OM | 13.3 | 12.7 | 12.6 | 12.0 | 0.58 | NS | NS | NS |
| NDF | 8.9 | 7.5 | 7.2 | 7.2 | 0.68 | .18 | NS | NS |
| Starch | 1.12 | 1.03 | 1.12 | .94 | 0.14 | NS | NS | NS |
| Ruminal turnover rate (%/h) | | | | | | | | |
| DM | 7.54 | 7.70 | 8.18 | 7.88 | 0.33 | NS | NS | NS |
| OM | 7.62 | 7.84 | 8.34 | 8.02 | 0.36 | NS | NS | NS |
| NDF | 3.23 | 3.45 | 3.76 | 3.59 | 0.19 | .10 | NS | NS |
| Starch | 36.59 | 40.02 | 37.10 | 39.69 | 4.7 | NS | NS | NS |

¹Hybrids used are Vitreous = Cargill 6409GQ and Floury = Syngenta NX7219.

TABLE 19
Effects of processing and corn hybrid on chewing behavior at Michigan State University.

| | Hybrid¹ | | | | | | | |
|---|---|---|---|---|---|---|---|---|
| | Vitreous | | Floury | | | P value | | |
| Item | nonproc | proc | nonproc | proc | SE | Endo | Proc | Interaction |
| Eating time (min) | | | | | | | | |
| /d | 255 | 236 | 258 | 259 | 6.6 | .07 | NS | .14 |
| /kg of DMI | 10.18 | 9.65 | 9.89 | 11.08 | 0.33 | .11 | NS | <.05 |
| /kg of NDF intake | 42.1 | 39.0 | 40.5 | 44.3 | 1.54 | NS | NS | <.05 |
| Ruminating time (min) | | | | | | | | |
| /d | 430.0 | 472.5 | 487.5 | 518.8 | 31.3 | .12 | NS | NS |
| /kg of DMI | 17.0 | 19.2 | 18.9 | 22.0 | 1.26 | .08 | .06 | NS |
| /kg of NDF intake | 69.8 | 78.0 | 77.8 | 87.2 | 4.69 | .09 | .08 | NS |
| Total chewing time (min) | | | | | | | | |
| /d | 685 | 708 | 745 | 778 | 31.5 | .06 | NS | NS |
| /kg of DMI | 27.1 | 28.9 | 28.9 | 33.0 | 1.26 | <.05 | <.05 | NS |
| /kg of NDF intake | 111.7 | 117.0 | 118.3 | 132.2 | 4.74 | <.05 | .08 | NS |
| Meal number (/d) | 8.25 | 9.13 | 8.88 | 8.75 | 0.44 | NS | NS | NS |
| Ruminating bouts (/d) | 12.0 | 12.3 | 12.5 | 12.6 | 0.58 | NS | NS | NS |

¹Hybrids used are Vitreous = Cargill 6409GQ and Floury = Syngenta NX7219.

As shown in Example II, the present invention establishes that the efficiency of milk production and total tract digestibility of starch is improved by use of floury endosperm silages and silage processing (rolling). The modest response to endosperm type in these studies likely reflected the fact that less than 15% of the dietary DM is comprised of the treatment starch. In accordance with the present invention, a synergistic response is possible when floury endosperm corn silage is combined with floury grain.

### EXAMPLE III

### Digestibility by Dairy Cows of Diets with Corn Silage Hybrids that Differed in Neutral Detergent Fiber Content

Silages:

A dual purpose hybrid (B52-B2, Syngenta Seeds Inc., Golden Valley, Minn.) and a hybrid bred to have high concentrations and in vitro digestibility of NDF (N48-V8, Syngenta Seeds, Inc.) are planted on Apr. 29, 1999 in similar fields located at the Ohio Agricultural Research and Development Center in Wooster. Seeding rate for both hybrids is 12,000 seeds/ha and agronomic practices are identical for each field. On Sep. 13, 1999, the dual purpose hybrid is harvested and put into a glass-lined steel silo (Harvestore, A.O. Smith Corp. Milwaukee Wis.). The following day, the other hybrid is harvested and put in a similar silo. Both hybrids are at the one-half milk stage at harvest, and are chopped at 0.95 cm theoretical length of cut using the same harvester without kernel processing. The silages remained undisturbed for about 4 mo.

Cows and Diets:

Eight multiparious Holstein cows at 174 DIM (SD=20) at the start of the experiment are randomly assigned to one of four treatment sequences in a 4×4 Latin square with 28 d periods. Cows are housed in individual tie stalls, fed once daily and milked twice daily. Diets are fed as TMR for 5 to 10% feed refusal per day. Milk weights are recorded electronically at each milking, and amount of feed offered and refused is measured daily. Cows are weighed approximately 4 h after feeding at the start of the experiment and then every 28 d. Body condition is scored (Wildman et al., 1982) at the start of the experiment and then every 28 d. During the last week of each period, six cows are moved into metabolism stalls for total collection of feces and urine for 4 d. A total of 24 (6 cows×4 periods) total collections are made; all cows are in the metabolism stalls three times and four observations per diet are made. All diets are fed in each collection period with two diets being fed to two different cows in each period. Methods used during the total collection period are described by Weiss and Wyatt (2000).

Four diets (Tables 1 and 2) are formulated that varied in corn silage hybrid and source and concentration of NDF. Diet 1 is formulated to contain 45% dual purpose corn silage (DPCS) and 46% concentrate (predominantly corn grain and soybean meal). Diet 2 is the same as diet 1 except that the high fiber corn silage (HFCS) is used. Diet 2 is formulated to have higher concentrations of total and forage NDF than diet 1. Diet 3 contained 33% HFCS and 58% concentrate (essentially the same concentrate as used in diet 1) and is formulated to provide the same concentrations of total and forage NDF as diet 1. Diet 4 contained 33% DPCS and 58% concentrate that contained soybean hulls to make dietary NDF concentration equal to that in diet 2.

Sampling and Analyses:

While the corn is being harvested, ears from ten random plants are collected from each field and hand-shelled, and the grain is analyzed for DM (100° C. overnight). Milk samples (a.m. and p.m.) are taken once each week and analyzed for fat and CP per approved procedures (AOAC, 1990) with a B2000 Infrared Analyzer (Bentley Instruments, Chaska Minn.) by Ohio DHI Cooperative, Inc. (Powell, Ohio). Silage samples are taken weekly and analyzed for DM by drying overnight at 100° C. to adjust diets for changes in DM. During the digestion trials, silages and concentrates are sampled daily and composited by period. The composited and weekly samples are dried at 60° C. and ground through a 2 mm screen (Wiley Mill, Arthur H. Thomas, Philadelphia, Pa.). These samples are analyzed for NDF (Procedure A; Van Soest et al., 1991) with sodium sulfite and amylase (Sigma A3306, Sigma Diagnostics, St. Louis, Mo.), ash, CP (N×6.25), ADF, and sulfuric acid lignin (AOAC, 1990), and starch, fermentation acids, and fatty acids (Weiss and Wyatt, 2000). The composited corn silage samples are analyzed for in vitro ruminal NDF digestibility (Goering and Van Soest, 1970). The four composited samples of each silage are incubated in rumen fluid and buffer in triplicate for either 30 or 48 h in a single run. Rumen fluid is collected from a single cow fed a diet of approximately 35% alfalfa silage, 15% corn silage, 29% ground corn, 6% soy hulls, 7% roasted whole soybeans, and 9% protein and mineral mix (DM basis). The composition data in Table 3, except for NDF and starch, are means of the four composited samples. In addition to the composited samples, weekly and biweekly samples are analyzed for NDF (n=16) and starch (n=12), respectively. During the third and fourth week of each period, two additional samples (on consecutive days) of the corn and alfalfa silages are collected, and particle size determined (Lammers et al., 1996). Samples from the middle screen and from the pan are collected (8 samples of each silage) and analyzed for starch as described above. During the digestion trial, daily samples of feces and oats are composited within cow and analyzed for ash, CP, NDF, starch, and fatty acids as described above. Daily milk and urine samples (pH kept <5 with HCl) are composited and analyzed for CP as described above. During the last week of each period (but not during the digestion trial), rumen fluid is obtained from each cow approximately 4 h after feeding via stomach tube. The samples are immediately acidified and frozen until analyzed for VFA by GLC (Supelco, 1975).

Statistical Analyses:

Mean (by cow) milk production, milk composition, and DMI are calculated from data collected during the last 2 wk of each period. The BW and BCS for each cow at the end of each period are analyzed statistically. Change in BW and BCS are calculated as the difference between the BW or BCS at the end of each period and the BW and BCS at the end of the preceding period. Apparent digestibility of nutrients is calculated as nutrient intake minus fecal nutrient output divided by intake. The TDN (% of DM) is calculated as concentration (% of DM) of digestible CP+digestible NDF+digestible nonfiber carbohydrate+(digestible fatty acids×2.25), where nonfiber carbohydrate=OM−CP−NDF−fatty acids. Production data are analyzed using Proc MIXED (SAS, 1999) for a model that included cow as a random variable (7 df), period (3 df), treatment (3 df), and error (22 df). Digestibility data are analyzed as an unbalanced Latin square; the model included cow as a random variable (7 df), period (3 df), treatment (3 df), and error (10 df). Means are separated using the PDIFF option of Proc MIXED when the treatment effect is significant (appropriate P values are shown in tables). Particle size data and in vitro digestibility of the two hybrids are compared with a t-test; different samples from the same silo (4 samples/hybrid) provided the experimental error.

Results:

The growing season is drier and hotter than normal (Table 4). Silage DM yields (not replicated) are 15.9 Mg/ha for the dual purpose hybrid and 16.1 Mg/ha for high fiber hybrid. Concentrations of CP, fatty acids, ash, and fermentation acids are similar for the two hybrids (Table 3). Mean NDF concentration is 6.6 percentage units higher and mean starch concentration is 4.4 percentage units lower for HFCS. Concentrations of NDF and starch in the silages are variable over time. In vitro NDF digestibility (30 and 48 h) is higher (P<0.05) for the HFCS. Particle size distribution of starch and DM are not different among hybrids (P>0.12).

Dry matter intake and yields of milk, 4% FCM, milk fat, and milk protein are not affected by treatment (Table 5). Milk fat percent tended to be lower (P<0.07) for cows fed the 33% HFCS diet. Based on changes in BW all cows are in positive energy balance, however, cows fed the 33% DPCS diet (contained soyhulls gained less (P<0.05) BW than cows fed 33% HFCS diet.

During the digestion trials, DMI is approximately 3.8× maintenance and is not affected by treatment (Table 6). The mass of wet feces excreted averaged 50.4 kg/d (SE=3.0) and urine excretion averaged 22.9 L/d (SE=1.9); neither is affected by treatment (data not shown). Digestibility of DM is not affected greatly by treatment but DM digestibility of the 33% DPCS diet tended to be lower than that of the 45% DPCS diet (Table 6). The two diets with the DPCS had lower (P<0.05) starch digestibility but tended to have higher (P<0.08) NDF digestibility as compared to the 33% HFCS diet. Measured TDN concentration of the 33% DPCS diet tended (P<0.09) to be lower than the TDN of the other three diets.

Diet NE_{L} concentration is calculated using: 1) the measured TDN concentration and equations (NRC, 1989, 2001); 2) the NRC (2001) model; and 3) summing NE_{L} used for maintenance, BW change, and milk production (NRC, 2001) and dividing by DMI. The three methods produced similar estimated NE_{L} concentrations (Table 6). The estimated NE_{L} content of the 33% DPCS diet generally is lower than the other diets. Estimated NE_{L} balance (NRC, 2001) are close to what is expected based on changes in BW (Table 5).

The molar proportion for ruminal propionate is higher (P<0.06) and acetate and the acetate to propionate ratio are lower (P<0.05) for the 33% HFCS diet compared to the two diets with the DPCS (Table 7).

Differences between the hybrids for concentrations of NDF and starch and in vitro NDF digestibility are as expected. Starch concentrations for both hybrids, however, are lower than expected. For a variety of hybrids and agronomic practices, reported starch concentrations for corn silage has ranged from about 26 to 37% of DM (Bal et al., 1997; Bal et al., 2000b; Weiss and Wyatt, 2000). The DPCS according to this Example III averaged 20.3% starch (Table 3). The probable reason for the low starch concentrations is the dry and hot conditions experienced during the growing season (Table 4). For four different hybrids, Frederick et al. (1990) reported significant reductions in kernel weight for drought-stressed corn compared with irrigated corn (43 vs 52% of whole plant DM).

Although DMI is not affected by treatment (Table 5), the diets differed in at least two factors that have been related to DMI (i.e., concentration and in vitro digestibility of NDF). Oba and Allen (1999b, 2000) reported that a 1-unit increase in in vitro NDF digestibility of brown midrib corn silage is associated with an increase in DMI of 0.15 kg/d. In this Example III, in vitro NDF digestibility of the HFCS is 4.7 percentage units higher than the DPCS (Table 3), which according to Oba and Allen (1999b, 2000) should have resulted in an increase in DMI of about 0.7 kg/d. That change is less than the standard error for DMI described in this Example.

The NDF content of the hybrids differed by 6.6 percentage units (Table 3) and total dietary NDF ranged from about 28 to 31% (Table 2). In most of the 15 papers reviewed by Allen (2000), DMI increased as the concentration of NDF in the diet decreased when dietary NDF is altered by changing the forage to concentrate ratio. In this Example III, diet NDF varied because of hybrid type, concentration of corn silage in the diet, and inclusion of a nonforage fiber source. Bal et al. (2000a) compared two hybrids that contained either 32.8 (low) or 39.2% NDF (normal). In diets with 50% forage, no difference in DMI between diets is observed (total dietary NDF is 24 and 27%), but in diets with about 60% forage, cows fed the low NDF silage had lower DMI than cows fed the normal NDF corn silage (Bal et al., 2000a). The HFCS had higher in vitro NDF digestibility (Table 3), but similar or lower in vivo NDF digestibilities (Table 6) are observed for diets containing HFCS. Corn silage provided between 42 and 70% of the total NDF in each diet (Table 2); therefore, smaller differences among treatments would be expected for in vivo, compared to in vitro, digestibility because of dilution. However, the differences in the amount of NDF provided by corn silage in the 45% DPCS diet and the 33% HFCS diet cannot explain the differences observed between in vitro and in vivo NDF digestibility. In vivo fiber digestibility is often reduced when excessive amounts of starch or NFC are fed (Llano and DePeters, 1985; Putnam and Loosli, 1959). The rumen VFA data (Table 7) and milk fat percent (Table 5) suggest that altered rumen conditions in cows fed the 33% HFCS diet caused the low in vivo NDF digestibility on that diet. This effect would not have occurred in vitro. The in vivo NDF digestibility for the 45% DPCS and 45% HFCS diets are more difficult to reconcile with in vitro data. Factors such as DMI and dilution of corn silage NDF, that could impact in vivo, but not in vitro, NDF digestibility are either not different or similar between those two treatments.

Significant correlations between in vitro DM digestibility and in vivo DM digestibility measured with sheep fed at approximate maintenance intakes have been observed (r=0.6 for unfermented corn plants; Aufrere et al., 1992 and r=0.8 for corn silage; De Boever et al., 1996). However, Oba and Allen (2000) reported a 9.4 percentage unit difference in 30 h in vitro NDF digestibility between a brown midrib corn silage and a control silage but no difference in vivo NDF digestibility. In another experiment with brown midrib corn silage, a 9.7 percentage unit difference in 30 h in vitro NDF digestibility between hybrids is found but in vivo NDF digestibility differed by only 2.2 unit (P<0.02) (Oba and Allen, 1999a). The disparity between in vitro and in vivo digestibilities in those studies is likely caused by differences in DMI. Both the data from in this Example III and data from brown midrib experiments raise concerns about using in vitro NDF digestibility as an estimate of in vivo digestibility by dairy cows fed mixed diets at productive intakes.

Table 6 shows improved starch digestibility by cows when fed the HFCS diets. Kernel DM is similar for the hybrids (64.5 and 63.6% for DPCS and HFCS, respectively), and probably is not different enough to affect starch digestibility. The majority of starch in all diets came from ground corn, rather than corn silage and the amount of starch provided by the corn silage varied among treatments (Table 2). On average, more starch came from the ground corn meal in diets with the HFCS than in diets with the DPCS. The starch in the HFCS could be more digestible than starch from the DPCS or the starch from the corn meal is more digestible than starch provided by corn silage, regardless of hybrid.

Overall responses to the different hybrids appeared to be related to the total forage concentration of the diet. When diets with 53.5% forage are fed, no differences are observed among hybrids in production, digestibility, rumen fermentation and energy balance except for higher starch and nonfiber carbohydrate digestibility for the diet with HFCS. More hybrid differences are observed when diets with 41.9% forage are fed. With a low forage diet, increasing dietary NDF with DPCS by including soy hulls increased milk fat, the molar proportion of ruminal acetate, the ratio of ruminal acetate to propionate, and NDF digestibility compared to the diet with additional NDF coming from the HFCS. The low forage, HFCS diet, however, provided more available energy to cows (this allowed for increased BW change) than did the low forage diet with DPCS and soy hulls.

Thus, a corn hybrid selected for higher concentrations of NDF and increased NDF digestibility had similar digestibility, calculated NE_{L} values, and supported similar production as a conventional dual purpose hybrid when fed in diets with 53.5% forage (44.6% corn silage and 8.9% alfalfa silage). The higher NDF concentration in HFCS did not reduce the available energy concentrations of the diets. Increased in vitro NDF digestibility, however, did not relate to increased DMI or increased in vivo NDF digestibility. In diets with 41.9% forage (33% corn silage, 8.9% alfalfa silage), the DPCS plus soy hull diet maintained milk fat percent and a more normal rumen fermentation than did the HFCS diet, but the HFCS diet had a higher available energy concentration and resulted in a more positive NE_{L} balance.

TABLE 1
Ingredient composition of the diets (% of DM).

| | Diet¹ | | | |
|---|---|---|---|---|
| | 45% | 45% | 33% | 33% |
| | DPCS | HFCS | HFCS | DPCS + SH |
| Alfalfa silage | 8.90 | 8.90 | 8.90 | 8.90 |
| Dual purpose corn silage | 44.60 | . . . | . . . | 33.00 |
| High fiber corn silage | . . . | 44.60 | 33.00 | . . . |
| Ground corn | 23.19 | 23.19 | 34.88 | 20.32 |
| Soybean hulls | . . . | . . . | . . . | 16.44 |
| Soybean meal, 44% CP | 17.32 | 17.32 | 17.11 | 15.23 |
| Tallow | 1.40 | 1.40 | 1.40 | 1.40 |
| Molasses, dried | 0.93 | 0.93 | 0.94 | 0.94 |
| Fish meal, menhaden | 0.42 | 0.42 | 0.42 | 0.42 |
| Blood meal, ring-dried | 0.42 | 0.42 | 0.42 | 0.42 |
| Limestone | 1.00 | 1.00 | 1.09 | 1.09 |
| Sodium bicarbonate | 0.79 | 0.79 | 0.80 | 0.80 |
| Mineral/vitamin premix² | 1.03 | 1.03 | 1.04 | 1.04 |

¹Diets: 45% DPCS = TMR with 45% dual purpose corn silage (DPCS) and 46% corn grain-based concentrate: 45% HFCS = TMR with 45% high fiber corn silage (HFCS) and 46% corn-grain based concentrate; 33% HFCS = TMR with 33% HFCS and 58% corn grain-based concentrate; 33% DPCS = TMR with 33% DPCS and 58% concentrate that contained soy hulls.
²Containcd 29.05% trace mineral salt, 23.27% dicalcium phosphate, 11.60% magnesium oxide, 9.70% Dynamate ® (IMC Global, Lake Forest, IL), 13.80% sodium sclenate premix (200 mg Se/kg), 0.46% copper sulfate, 0.46% zinc oxide, 2.00% vitamin A premix (30,000 IU/g), 4.14% vitamin D premix (3000 IU/g), and 5.52% vitamin E premix (44 IU/g).

TABLE 2
Nutrient composition of the diets (DM basis).

| | Diet¹ | | | |
|---|---|---|---|---|
| | 45% | | | 33% |
| | DPCS | 45% HFCS | 33% HFCS | DPCS + SH |
| DM, % as fed | 62.8 | 62.8 | 68.3 | 69.0 |
| CP, % | 17.5 | 17.5 | 17.3 | 17.3 |
| NDF, % | 29.3 | 31.4 | 27.3 | 32.9 |
| Forage NDF, % | 23.6 | 25.7 | 20.1 | 18.5 |
| Corn silage NDF, | 64.5 | 69.6 | 59.2 | 42.5 |
| % of NDF | | | | |
| Starch, % | 25.4 | 22.3 | 29.5 | 23.4 |
| Corn silage starch, | 36.2 | 27.8 | 15.6 | 29.1 |
| % of starch | | | | |
| NFC², % | 43.9 | 41.4 | 45.8 | 40.2 |
| Fatty acids, % | 4.6 | 4.4 | 4.6 | 4.4 |
| Ca, % | 0.80 | 0.80 | 0.83 | 0.84 |
| P, % | 0.38 | 0.38 | 0.37 | 0.38 |
| Mg, % | 0.25 | 0.25 | 0.24 | 0.26 |

¹Diets: 45% DPCS = TMR with 45% dual purpose corn silage (DPCS) and 46% corn grain-based concentrate: 45% HFCS = TMR with 45% high fiber corn silage (HFCS) and 46% corn-grain based concentrate; 33% HFCS = TMR with 33% HFCS and 58% corn grain-based concentrate; 33% DPCS = TMR with 33% DPCS and 58% concentrate that contained soy hulls.
²Nonfiber carbohydrates = OM − CP − NDF − fatty acids (all values as percent of DM).

TABLE 3
Nutrient composition of the silages fed during the trial (DM basis).

| | Corn silage hybrid¹ | | |
|---|---|---|---|
| | DPCS | HFCS | Alfalfa silage |
| DM, % as-fed | 38.0 | 38.1 | 53.1 |
| OM, % | 96.0 | 95.9 | 90.0 |
| CP, % | 8.6 | 8.3 | 20.4 |
| NDF, % | 42.4 | 49.0 | 47.8 |
| ADF, % | 24.3 | 26.0 | 36.8 |
| Lignin, % | 2.5 | 2.6 | 7.0 |
| Starch, % | 20.3 | 15.9 | 1.4 |
| Fatty acids, % | 3.0 | 2.6 | 2.0 |
| IVNDFD-30 h², % | 35.4* | 40.1* | ND |
| IVNDFD-48 h², % | 41.5** | 46.3** | ND |
| pH | 3.86 | 3.95 | 5.33 |
| Acetic acid, % | 2.3 | 2.0 | 2.0 |
| Butyric acid, % | 0.2 | 0.2 | 0.2 |
| Lactic acid, % | 4.5 | 4.1 | 2.3 |
| Particle size distribution | | | |
| of DM³, % of total | | | |
| Top | 1.2 | 2.1 | 8.3 |
| Middle | 44.5 | 46.1 | 45.0 |
| Pan | 55.3 | 51.8 | 46.7 |
| Particle size distribution | | | |
| of starch³ % of total | | | |
| Middle | 69.4 | 64.8 | ND |
| Pan | 30.6 | 35.2 | ND |

*P < 0.05;
**P < 0.01.
¹DPCS = dual purpose corn silage; HFCS = high fiber corn silage.
²In vitro rumen digestibility of NDF after 30 or 48 h incubation. Standard error for four separate in vitro runs per silage is 1.3 (30 h) and 0.8 (48 h). ND = not determined.
³Measured using a two screen sieve plus a pan (Lammers et al., 1996).

TABLE 4
Climatic conditions during the 1999 growing season¹.

| | |
|---|---|
| Temperature, ° C. | Precipitation, mm |

| Month | Mean | Deviation² | Total | Deviation² |
|---|---|---|---|---|
| March | 1.1 | −2.3 | 45 | −29 |
| April | 10.6 | 2.0 | 159 | 81 |
| May | 16.1 | 1.3 | 61 | −41 |
| June | 20.6 | 1.2 | 34 | −55 |
| July | 23.3 | 1.7 | 113 | 10 |
| August | 19.4 | −0.9 | 86 | −8 |
| September | 16.1 | −0.8 | 61 | −21 |

¹Data collected from a weather station located approximately 2 km from the plots.
²Deviation = actual minus 30-yr monthly average.

TABLE 5
Production by cows fed diets containing different corn silage hybrids.

| | Diet¹ | | | | |
|---|---|---|---|---|---|
| | 45% | 45% | 33% | 33% | |
| | DPCS | HFCS | HFCS | DPCS + SH | SEM |
| Dry matter intake, | 23.9 | 23.7 | 23.7 | 23.4 | 0.76 |
| kg/d | | | | | |
| Milk, kg/d | 33.3 | 34.0 | 35.5 | 33.8 | 0.97 |
| 4% FCM, kg/d | 33.3 | 33.3 | 33.5 | 33.6 | 1.36 |
| Milk fat, % | 3.93^{a} | 3.82^{ab} | 3.53^{b} | 3.95^{a} | 0.17 |
| kg/d | 1.33 | 1.32 | 1.29 | 1.34 | 0.07 |
| Milk protein, % | 3.31 | 3.27 | 3.30 | 3.27 | 0.02 |
| kg/d | 1.12 | 1.11 | 1.17 | 1.11 | 0.04 |
| BW, kg | 673 | 675 | 669 | 666 | 3 |
| BW change, kg/28 d | 15^{ab} | 14^{ab} | 17^{a} | 4^{b} | 4.5 |
| BCS | 3.0 | 3.0 | 2.9 | 2.9 | 0.06 |
| BCS change | 0.1 | 0.0 | 0.1 | 0.1 | 0.07 |
| (unit/28 d) | | | | | |
| NE_{L} balance², | 3.7^{x} | 2.7^{y} | 3.2^{x} | 2.1^{y} | 0.96 |
| Mcal/g | | | | | |

^{a,b}Means differ (P < 0.07).
^{x,y}Means differ (P < 0.09).
¹Diets: 45% DPCS = TMR with 45% dual purpose corn silage (DPCS) and 46% corn grain-based concentrate: 45% HFCS = TMR with 45% high fiber corn silage (HFCS) and 46% corn-grain based concentrate; 33% HFCS = TMR with 33% HFCS and 58% corn grain-based concentrate; 33% DPCS = TMR with 33% DPCS and 58% concentrate that contained soy hulls.
²Calculated by entering diet composition and individual BW, milk production and composition and DMI for each cow (within a period) into the NRC (2001) model.

TABLE 6
Intake during digestion trial and digestibility of nutrients in diets with
different corn silage hybrids (DM basis).

| | Diet¹ | | | | | |
|---|---|---|---|---|---|---|
| | 45% | 45% | 33% | 33% | | |
| | DPCS | HFCS | HFCS | DPCS + SH | SEM | P< |
| DMI, kg/d | 24.3 | 24.3 | 23.8 | 24.8 | 1.16 | NS |
| Digestibility, % | | | | | | |
| DM | 65.7^{a} | 65.0^{ab} | 65.5^{ab} | 63.9^{b} | 0.67 | 0.10 |
| OM | 66.8 | 66.3 | 66.7 | 65.2 | 0.64 | NS |
| CP | 67.1^{a} | 66.0^{ab} | 66.6^{a} | 63.6^{b} | 0.65 | 0.06 |
| NDF | 42.2^{a} | 39.8^{ab} | 35.3^{b} | 41.7^{a} | 1.89 | 0.08 |
| Starch | 88.5^{c} | 91.8^{ab} | 93.1^{a} | 90.2^{bc} | 0.70 | 0.05 |
| NFC | 82.1^{b} | 85.2^{a} | 86.8^{a} | 82.0^{b} | 0.72 | 0.05 |
| Fatty acids | 69.2 | 75.9 | 74.3 | 73.7 | 3.2 | NS |
| TDN | 67.2^{a} | 67.0^{b} | 67.7^{a} | 65.9^{b} | 0.62 | 0.09 |
| Estimated NE_{L} | | | | | | |
| Mcal/kg | | | | | | |
| Calculated | 1.62 | 1.61 | 1.63 | 1.57 | . . . | . . . |
| from TDN² | | | | | | |
| NRC (2001)³ | 1.64 | 1.63 | 1.65 | 1.63 | . . . | . . . |
| Energy | 1.59 | 1.60 | 1.62 | 1.55 | . . . | . . . |
| use/DMI⁴ | | | | | | |

^{a,b,c}Means in a row with unlike superscripts differ by stated P value (the P value is the probability associated with treatment main effect).
NS = P > 0.15
¹Diets: 45% DPCS = TMR with 45% dual purpose corn silage (DPCS) and 46% corn grain-based concentrate: 45% HFCS = TMR with 45% high fiber corn silage (HFCS) and 46% corn-grain based concentrate; 33% HFCS = TMR with 33% HFCS and 58% corn grain-based concentrate; 33% DPCS = TMR with 33% DPCS and 58% concentrate that contained soy hulls.
²NE_{L} calculated from TDN: Digestible energy (DE) = TDN × 0.04409; Metabolizable energy (ME) = 1.01 × DE − 0.45; NE_{L} = 0.709 × ME − 0.19 (NRC, 1989, 2001).
³Calculated (NRC, 2001) using individual cow data and actual diet composition.
⁴Sum of NE_{L} requirements for maintenance, milk production and BW change (NRC, 2001) divided by DMI.

TABLE 7
Ruminal VFA (moles/100 moles of total VFA) for cows fed different
corn silage hybrids.

| | Diet¹ | | | | | |
|---|---|---|---|---|---|---|
| | 45% | 45% | 33% | 33% | | |
| | DPCS | HFCS | HFCS | DPCS + SH | SEM | P< |
| Acetate (A) | 59.6^{a} | 59.1^{ab} | 57.2^{b} | 60.4^{a} | 1.15 | 0.05 |
| Propionate (P) | 21.8^{b} | 22.6^{ab} | 24.6^{a} | 21.0^{b} | 0.96 | 0.06 |
| Butyrate | 14.5 | 14.3 | 13.8 | 14.6 | 0.62 | NS |
| A:P | 2.76^{ab} | 2.67^{ab} | 2.41^{b} | 3.02^{a} | 0.19 | 0.04 |

^{a,b}Means with unlike superscript in same row differ by stated P value (the P value is the probability associated with treatment main effect).
NS = P > 0.15.
¹Diets: 45% DPCS = TMR with 45% dual purpose corn silage (DPCS) and 46% corn grain-based concentrate: 45% HFCS = TMR with 45% high fiber corn silage (HFCS) and 46% corn-grain based concentrate; 33% HFCS = TMR with 33% HFCS and 58% corn grain-based concentrate; 33% DPCS = TMR with 33% DPCS and 55% concentrate that contained soy hulls.

### REFERENCES

Allen, M. S. Corn silage processing: it's a matter of faith. Michigan Dairy Review November, 1998. Department of Animal Science, Michigan State University, East Lansing.
Allen, M. S. 2000. Effects of diet on short-term regulation of feed intake by lactating dairy cows. J. Dairy Sci. 83:598-1624.
Association of Official Analytical Chemists. 1990. Official Methods of Analysis. Vol. I. 15^{th} ed. AOAC, Arlington, Va.
Aufrere, J., D. Graviou, C. Demarquilly, J. Andrieu, J. C. Emile, R. Giovanni, and P. Maupetit. 1992. Estimation of organic matter digestibility of whole maize plants by laboratory methods. Anim Feed Sci Tech 36:187-204.
Bal, M. A. J. G. Coors, and R. D. Shaver. 1997. Corn silage maturity effects on intake and milk production by dairy cows. J. Anim. Sci. 75 (Suppl. 1):96.
Bal, M. A., R. D. Shaver, K. J. Shinners, and L. D. Satter. 1998a. Effects of mechanical processing on the utilization of whole-plant corn silage by lactating dairy cows. J. Dairy Sci. 81 (Suppl. 1): 334 (abstr.)
Bal, M. A., J. G. Coors, and R. D. Shaver. 1997. Impact of the maturity of corn for use as silage in the diets of dairy cows on intake, digestion, and milk production. J. Dairy Sci. 80:2497-2503.
Bal, M. A., R. D. Shaver, H. Al-Jobeile, J. G. Coors, and J. G. Lauer. 2000a. Corn silage hybrid effects on intake, digestion, and milk production by dairy cows. J. Dairy Sci. 83:2849-2858.
Bal, M. A., R. D. Shaver, K. J. Shinners, J. G. Coors, J. G. Lauer, R. J. Straub, and R. G. Koegel. 2000b. Stage of maturity, processing, and hybrid effects on ruminal in situ disappearance of whole-plant corn silage. Anim. Feed Sci. Technol. 86:83-94.
Cox, W. J., J. H. Cherney, D. J. R. Cherney, and W. D. Pardee. 1994. Forage quality and harvest index of corn hybrids under different growing conditions. Agron. J. 86:277-282.
Dado, R. G., and R. W. Briggs. 1996. Ruminal starch digestibility of grain from high-lysine corn hybrids harvested after black layer. J. Dairy Sci. 79 (Suppl. 1):213.
De Boever, J. L., B. G. Cottyn, D. L. D. Brabander, J. M. Vanacker, and C. V. Boucque. 1996. Prediction of the feeding value of maize silages by chemical parameters, in vitro digestibility and NIRS. Anim. Feed Sci. Technol 66:211-222.
Frederick, J. R., F. E. Below, and J. D. Hesketh. 1990. Carbohydrate, nitrogen and dry matter accumulation and partitioning of maize hybrids under drought stress. Annals of Botany 66:407-415.
Goering, H. K., and P. J. Van Soest. 1970. Forage Fiber Analysis (Apparatus, Reagents, Procedures, and Some Applications). Agric. Handbook No. 379. ARS-USDA, Ishington, D.C.
Harrison, J. H., L. Johnson, R. Riley, S. Xu, K. Loney, C. W. Hunt, and D. Sapienza. 1996a. Effects of harvest maturity of whole plant corn silage on milk production and component yield, and passage of corn grain and starch into feces. J. Dairy Sci. 79 (Suppl. 1): 149. (Abstr.)
Herrera-Saldana, R. and J. T. Huber. 1989. J Dairy Sci. 72:1477.
Johnson, L., J. H. Harrison, C. Hunt, K. Shinners, C. G. Doggett, and D. Sapienza. 1999. Nutritive value of corn silage as affected by maturity and mechanical processing: a contemporary review. J. Dairy Sci. 82:2813.
Lammers, B. P., D. R. Buckmaster, and A. J. Heinrichs. 1996. A simple method for the analysis of particle sizes of forage and total mixed rations. J. Dairy Sci. 79:922-928.
Llano, C. A., and E. J. DePeters. 1985. Apparent digestibilities of diets varying in ratios of forage to concentrate and quality of forage at two intakes by dairy cows. J. Dairy Sci. 68:1189-1197.
National Research Council. 2001. Nutrient Requirements of Dairy Cattle. Natl. Acad. Press, Ishington D.C.
Oba, M. and M. S. Allen. 2000. Effects of brown midrib 3 mutation in corn silage on productivity of dairy cows fed two concentrations of dietary NDF:2. chewing activities. J. Dairy Sci. 83:1342
Oba, M., and M. S. Allen. 1999a. Effects of brown midrib 3 mutation in corn silage on dry matter intake and productivity of high yielding dairy cows. J. Dairy Sci. 82:135-142.
Oba, M., and M. S. Allen. 1999b. Evaluation of the importance of the digestibility of neutral detergent fiber from forage: Effects on dry matter intake and milk yield of dairy cows. J. Dairy Sci. 82:589-596
Philippeau, C. and B. Michalet-Doreau. 1998. Influence of genotype and ensiling of corn grain on in situ degradation of starch in the rumen. J. Dairy Sci. 81:2178-2184.
Philippeau, C. and B. Michalet-Doreau. 1997. Influence of genotype and stage of maturity of maize on rate of ruminal starch degradation. Anim. Feed Sci. Technol. 68:25-35.
Putnam, P. A., and J. K. Loosli. 1959. Effect of feeding different ratios of roughage to concentrate upon milk production and digestibility of the ration. J. Dairy Sci. 42:1070-1078.
SAS. 1998. SAS User's Guide: Statistics. SAS Inst. Inc., Cary, N.C.
Satter, L. D., Z. Wu, V. R. Moreira, M. A. Bal, and R. D. Shaver. 1999. Processing corn silage. Proc. 24^{th} Annual Minnesota Forage Conf., Feb. 10-11, 1999, Rochester, Minn.
Supelco. 1975. GC separation of VFA C2-C5, Bulletin 749D, Supelco, Inc., Bellefonte, Pa.
Tine, M. A., K. R. McLeod, R. A. Erdman, and R. L. Baldwin VI. 2001. Effects of brown midrib corn silage on the energy balance of dairy cattle. J. Dairy Sci. 84:885-895.
Van Soest, P. J., J. B. Robertson, and B. A. Lewis. 1991. Methods for dietary fiber, neutral detergent fiber, and nonstarch polysaccharides in relation to animal nutrition. J. Dairy Sci. 74:3583.
Weiss, W. P., and D. J. Wyatt. 2000. Effect of oil content and kernel processing of corn silage on digestibility and milk production by dairy cows. J. Dairy Sci. 83:351-358.
Wildman, E. E., G. M. Jones, P. E. Wagner, R. C. Boman, H. F. Troutt, Jr., and T. N. Lesch. 1982. A dairy cow condition scoring system and its relationship to selected characteristics. J. Dairy Sci. 65:495.

What is claimed is:

1) A method for enhancing ruminal production comprising the steps of:
a) selecting a corn hybrid for use as supplemental grain;
b) preparing said supplemental grain comprising said corn hybrid;
c) determining the endosperm type of said corn hybrid;
d) selecting silage to combine with said supplemental grain to form a feed ration for a ruminant; and
e) determining the amounts of said silage and said supplemental grain for said feed ration, wherein when said feed ration is consumed by the ruminants, said amounts optimize the ruminal environment for starch degradation and energy utilization.

2) A method for enhancing ruminant production according to claim 1, wherein said supplemental grain is prepared from said hybrid corn having a high moisture content.

3) A method for enhancing ruminant production according to claim 1, wherein said supplemental grain is prepared using said corn hybrid having dry grain.

4) A method for enhancing ruminant production according to clam 1, wherein said supplemental grain is prepared using said hybrid corn having been steam flaked.

5) A method for enhancing ruminant production according to claim 1, wherein said supplemental grain is prepared based on particle size.

6) A method for enhancing ruminant production according to claim 1, wherein the endosperm of said corn hybrid is floury.

7) A method for enhancing ruminant production according to claim 1, wherein the endosperm of said corn hybrid is vitreous.

8) A method for enhancing ruminant production comprising the steps of:
a) selecting a first corn hybrid for floury endosperm type for use as supplemental corn grain;
b) preparing a supplemental corn grain comprising said first corn hybrid;
c) selecting a second corn hybrid with high NDF, low starch, content for use as corn silage;
d) preparing corn silage comprising said second corn hybrid; and
e) combining said supplemental grain, said corn silage, and other components to form a feed ration, wherein said supplemental grain and said corn silage are combined in amounts to optimize the ruminal environment for starch degradation and energy utilization.

9) A method for enhancing ruminant production according to claim 8, wherein said corn hybrid selected for use as corn silage has a low NDF, high starch, content.

10) A method for enhancing ruminant production according to claim 8, wherein said corn hybrid selected for use as corn silage has high NDF digestibility.

11) A method for enhancing ruminant production according to claim 1, wherein said ruminant is a dairy cow.

12) A method for enhancing ruminant production according to claim 1, wherein said ruminant is a steer.

13) A method for enhancing ruminant production according to claim 1, wherein said corn hybrid is corn hybrid N48-V8.

14) A method for enhancing ruminant production according to claim 1, wherein said supplemental grain is comprised of the seed of corn hybrid NX7219.
